SEQUENCE LISTING (15 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 685 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 588 aa, Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 685 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAR

SEQ ID NO: 6 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 7 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 8 - protein, 1271 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 683 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRAR

SEQ ID NO: 10 - protein, 588 aa, Severe acute respiratory syndrome coronavirus 2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 12 - protein, 8 aa, Severe acute respiratory syndrome coronavirus 2
VDEAGSKS

SEQ ID NO: 13 - protein, 7 aa, Severe acute respiratory syndrome coronavirus 2
DEAGSKS

SEQ ID NO: 14 - protein, 5 aa, Severe acute respiratory syndrome coronavirus 2
VLVVL

SEQ ID NO: 15 - protein, 8 aa, Severe acute respiratory syndrome coronavirus 2
PFTINCQE

(19) United States
(12) Patent Application Publication
(10) Pub. No.: US 2023/0364221 A1
(43) Pub. Date: Nov. 16, 2023

(54) VIRAL VACCINE COMPOSITIONS FOR INOCULATING A SUBJECT AGAINST A CORONAVIRUS, AN INFLUENZA VIRUS, OR BOTH

(71) Applicant: Atossa Therapeutics, Inc., Seattle, WA (US)

(72) Inventor: Steven C. QUAY, Seattle, WA (US)

(21) Appl. No.: 18/029,812

(22) PCT Filed: Oct. 14, 2021
(86) PCT No.: PCT/US2021/055077

§ 371 Date: Mar. 31, 2023

Related U.S. Application Data
(60) Provisional application No. 63/092,045, filed on Oct. 15, 2020.

(51) Int. Cl. A61K 39/215; A61K 47/10; A61K 39/145; A61K 47/12; A61P 37/04; A61K 47/18

(52) U.S. Cl. CPC A61K 39/215; A61K 47/10; A61K 39/145; A61K 47/12; A61P 37/04; A61K 47/186; A61K 2039/543

(57) ABSTRACT

Described herein are viral vaccine compositions for inoculating a subject against a coronavirus, an influenza virus, or both. The viral vaccine compositions may contain coronavirus antigens, influenza virus antigens, or both to elicit a sustained immune response in the subject. A viral vaccine may be formulated for nasal delivery. Also described herein are methods of administering viral vaccine compositions to a subject to prevent a coronavirus infection, influenza, or both. The viral vaccine compositions may be administered intranasally and may elicit an antigen-specific mucosal immune response in the subject.

### CROSS-REFERENCE

The present application claims the benefit of U.S. Provisional Application No. 63/092,045, entitled “VIRAL VACCINE COMPOSITIONS FOR INOCULATING A SUBJECT AGAINST A CORONAVIRUS, AN INFLUENZA VIRUS, OR BOTH,” filed on Oct. 15, 2020, which application is herein incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Oct. 14, 2021, is named 109559-694101_SL.txt and is 65,883 bytes in size.

### BACKGROUND

In the months since its identification, coronavirus disease 2019 (COVID-19), caused by the severe acute respiratory syndrome coronavirus 2 (SARS-CoV-2) virus, has killed millions of people worldwide. Additionally, influenza is estimated to kill between 250,000 and 500,000 people annually. Development of a vaccine capable of eliciting sustained immunity to viruses in the respiratory tract has proved challenging in the ongoing COVID-19 pandemic. There is a need for a viral vaccine that convers immunity to SARS-CoV-2 and protection against respiratory viral infection that can be rapidly distributed and administered.

### SUMMARY

In various aspects, the present disclosure provides a composition comprising: a first coronavirus spike protein in a prefusion complex, and a second coronavirus spike in a protein postfusion complex; wherein the composition is formulated for nasal delivery.

In some aspects, the composition further comprises a STING pathway agonist encapsulated in a lipid nanoparticle. In some aspects, the STING pathway agonist comprises a cyclic dinucleotide. In some aspects, the cyclic dinucleotide comprises cyclic guanosine monophosphate-adenosine monophosphate. In some aspects, the liposome is negatively charged. In some aspects, the liposome comprises an average zeta potential of no greater than 0 mV. In some aspects, the liposome has a mean diameter of no less than 30 nm and no greater than 300 nm.

In some aspects, the postfusion complex comprises one or more N-linked glycans. In some aspects, the first coronavirus spike protein comprises one or more N-linked glycans linked to amino acid residues N1098, N1134, N1158, N1173, or N1194, with respect to SEQ ID NO: 1, or combinations thereof. In some aspects, the second coronavirus spike protein comprises one or more N-linked glycans linked to amino acid residues N1098, N1134, N1158, N1173, or N1194, with respect to SEQ ID NO: 1, or combinations thereof. In some aspects, the first coronavirus spike protein comprises a sequence having at least 90% sequence identity to any one of SEQ ID NO: 1-SEQ ID NO: 5 or SEQ ID NO: 8-SEQ ID NO: 10. In some aspects, the second coronavirus spike protein comprises a sequence having at least 90% sequence identity to any one of SEQ ID NO: 1-SEQ ID NO: 5 or SEQ ID NO: 8-SEQ ID NO: 10.

In various aspects, the present disclosure provides a composition comprising: a coronavirus antigen, an influenza A antigen, an influenza B antigen, or both, and a STING pathway agonist encapsulated in a lipid nanoparticle, wherein the composition is formulated for nasal delivery.

In some aspects, the coronavirus antigen is selected from the group consisting of a spike protein, an M protein, an E protein, or an ORF8 protein. In some aspects, the spike protein is in a prefusion complex. In some aspects, the spike protein is in a postfusion complex. In some aspects, the spike protein comprises one or more N-linked glycans. In some aspects, the spike protein comprises one or more N-linked glycans linked to amino acid residues N1098, N1134, N1158, N1173, or N1194, with respect to SEQ ID NO: 1, or combinations thereof. In some aspects, the spike protein comprises a sequence having at least 90% sequence identity to any one of SEQ ID NO: 1-SEQ ID NO: 5 or SEQ ID NO: 8-SEQ ID NO: 10. In some aspects, the M protein comprises a sequence having at least 90% sequence identity to SEQ ID NO: 6. In some aspects, the E protein comprises a sequence having at least 90% sequence identity to SEQ ID NO: 7. In some aspects, the influenza A antigen is from an influenza A subtype selected from H1N1, H3N2, or a combination thereof. In some aspects, the influenza B antigen is from an influenza B lineage selected from Victoria, Yamagata, or a combination thereof.

In some aspects, the STING pathway agonist comprises a cyclic dinucleotide. In some aspects, the cyclic dinucleotide comprises cyclic guanosine monophosphate-adenosine monophosphate. In some aspects, the liposome is negatively charged. In some aspects, the liposome comprises an average zeta potential of no greater than 0 mV. In some aspects, the liposome has a mean diameter of no less than 30 nm and no greater than 300 nm.

In some aspects, the composition further comprises a preservative. In some aspects, the composition further comprises a buffer. In some aspects, the composition further comprises a humectant. In some aspects, the composition comprises a viscosity of no more than 1000 cPs.

In various aspects, the present disclosure provides a composition comprising: a coronavirus antigen, a STING pathway agonist encapsulated in a lipid nanoparticle, a preservative, a buffer, and a humectant; wherein the composition comprises a viscosity of no more than 1000 cPs, and wherein the composition is formulated for nasal delivery.

In some aspects, the coronavirus antigen is selected from the group consisting of a spike protein, an M protein, or an E protein. In some aspects, the spike protein is in a prefusion complex. In some aspects, the spike protein is in a postfusion complex. In some aspects, the spike protein comprises one or more N-linked glycans. In some aspects, the spike protein comprises one or more N-linked glycans linked to amino acid residues N1098, N1134, N1158, N1173, or N1194, with respect to SEQ ID NO: 1, or combinations thereof. In some aspects, the spike protein comprises a sequence having at least 90% sequence identity to any one of SEQ ID NO: 1-SEQ ID NO: 5 or SEQ ID NO: 8-SEQ ID NO: 10. In some aspects, the M protein comprises a sequence having at least 90% sequence identity to SEQ ID NO: 6. In some aspects, the E protein comprises a sequence having at least 90% sequence identity to SEQ ID NO: 7.

In some aspects, the STING pathway agonist comprises a cyclic dinucleotide. In some aspects, the cyclic dinucleotide comprises cyclic guanosine monophosphate-adenosine monophosphate. In some aspects, the liposome is negatively charged. In some aspects, the liposome comprises an average zeta potential of no greater than 0 mV. In some aspects, the liposome has a mean diameter of no less than 30 nm and no greater than 300 nm.

In some aspects, the composition further comprises an influenza A antigen. In some aspects, the influenza A antigen is from an influenza A subtype selected from H1N1, H3N2, or a combination thereof. In some aspects, the composition further comprises an influenza B antigen. In some aspects, the influenza B antigen is from an influenza B lineage selected from Victoria, Yamagata, or a combination thereof.

In some aspects, the humectant is selected from the group consisting of sorbitol, propylene glycol, and glycerin, and combinations thereof. In some aspects, the humectant is propylene glycol. In some aspects, the buffer is selected from the group consisting of citric acid, sodium citrate, monopotassium phosphate, disodium phosphate, potassium biphthalate, sodium hydroxide, sodium acetate, acetic acid, and combinations thereof. In some aspects, the buffer comprises citric acid and sodium citrate. In some aspects, the preservative is selected from the group consisting of benzyl alcohol, parabens thimerosal, chlorobutanol, benzethonium chloride, and benzalkonium chloride, and combinations thereof. In some aspects, the composition comprises a pH of no less than 4 and no greater than 6. In some aspects, the composition is sterile, as measured by a U.S. Pharmacopeia <61> method. In some aspects, the composition comprises an impurity at a concentration of no more than 0.10% relative to the coronavirus protein or no more than 1.0 mg per day intake. In some aspects, the impurity comprises a synthesis-related impurity, a degradation impurity, a heavy metal, or a combination thereof.

In some aspects, the composition is capable of preventing a viral infection in a subject nasally administered the composition. In some aspects, the composition is capable of reducing the severity of a viral infection in a subject nasally administered the composition. In some aspects, the viral infection is a coronavirus infection, an influenza virus infection, or a combination thereof.

In various aspects, the present disclosure provides a method of administering a viral vaccine against a virus to a subject, the method comprising, infusing a nose of the subject with a composition of the present disclosure in a spray plume, wherein the amount of the composition delivered in a spray plume contains no less than 75% and no more than 125% of a target spray volume.

In various aspects, the present disclosure provides a method of immunizing a subject against a virus, the method comprising nasally administering to the subject a composition of the present disclosure, thereby immunizing the subject.

The method of claim 63 or claim 64, wherein administering the composition to the subject increases an immunity to the virus in the subject.

In some aspects, administering the composition to the subject prevents an infection caused by the virus. In some aspects, administering the composition to the subject reduces a severity of an infection caused by the virus. In some aspects, reducing the severity of the infection comprises reducing a risk of hospitalization. In some aspects, reducing the severity of the infection comprises increasing a likelihood that the infection is asymptomatic. In some aspects, reducing the severity of the infection comprises decreasing a severity of a respiratory symptom caused by the virus. In some aspects, administering the composition to the subject reduces a viral load of the virus in the subject. In some aspects, administering the composition to the subject increases a mucosal immunity to the virus in the subject. In some aspects, administering the composition to the subject increases production of antibodies against the virus in the subject. In some aspects, the antibodies are produced in a lung of the subject.

In some aspects, the virus is a coronavirus, an influenza virus, or a combination thereof. In some aspects, the coronavirus is SARS-CoV-2. In some aspects, the SARS-CoV-2 comprises an alpha variant, a beta variant, a gamma variant, a delta variant, iota variant, kappa variant, or a combination thereof. In some aspects, the influenza virus is an influenza A, an influenza B, or a combination thereof.

In some aspects, the method comprises: providing an actuator comprising an actuator tip; producing an aerosol comprising droplets of the pharmaceutical composition from the actuator tip; and dispensing the aerosol into a nose of the subject. In some aspects, the method comprises producing a spray pattern ellipticity ratio of no less than 1.0 to not more than 1.4. In some aspects, the method comprises producing a droplet size distribution such that no more than about 5% of the aerosol volume forms droplets that are less than about 10 μm in diameter. In some aspects, the method comprises producing a droplet size distribution such that at least 0.4% of the aerosol volume forms droplets that are less than about 10 μm in diameter. In some aspects, the method comprises producing a droplet size distribution such that no more than about 50% of the aerosol volume forms droplets that are less than about 26.9 μm in diameter. In some aspects, the method comprises producing a droplet size distribution such that a diameter of droplet for which 50% of the aerosol volume forms droplets of smaller diameter is no less than 25 μm and not more than 75 μm. In some aspects, the method comprises producing a droplet size distribution such that a diameter of droplet for which 10% of the aerosol volume forms droplets of smaller diameter is no less than 15 μm and not more than 35 μm. In some aspects, the method comprises producing a droplet size distribution such that a diameter of droplet for which 90% of the aerosol volume forms droplets of smaller diameter is no less than 70 μm and not more than 150 p.m. In some aspects, the method comprises producing a spray pattern with a major axis of at least 25 mm and not more than 40 mm and a minor axis of at least 25 mm and not more than 40 mm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a spray pattern (top) and a plume geometry (bottom) of a first device for nasal delivery of viral vaccine.

FIG. 2 illustrates a spray pattern (top) and a plume geometry (bottom) of a second device for nasal delivery of viral vaccine.

### DETAILED DESCRIPTION

The present disclosure provides compositions and methods for prevention of viral infections. The compositions of the present disclosure may be administered to a subject as a viral vaccine in order to inoculate the subject against one or more viruses. In some embodiments, a viral vaccine of the present disclosure may inoculate the subject against a coronavirus (e.g., SARS-CoV-2, SARS-CoV, or MERS-CoV), an influenza virus (e.g., influenza A or influenza B), or combinations thereof. The compositions of the present disclosure may be formulated for nasal delivery and delivered to a subject intranasally. Intranasal delivery of the viral vaccines of the present disclosure may be well-suited for inoculation against respiratory viruses (e.g., coronaviruses and influenza viruses) due to mucosal immune response following intranasal administration relative to other delivery routes (e.g., injection or oral administration). Administering a vaccine via the nasal passage may result in an enhanced mucosal immune response, for example at mucus membranes in the respiratory tract, relative to other methods of administration (e.g., injection or oral administration). Enhanced mucosal immunity may lead to improved immunity at the site of infection (e.g., the lungs) and may reduce the risk of infection or reduce the severity of respiratory symptoms in the event of infection.

Intranasal vaccine delivery may provide numerous advantages over conventional vaccines administered via injection (e.g., intramuscular, subcutaneous, or intradermal injection). For example, fear of needles, identified in nearly one in four adults, may be an underlying cause of vaccine hesitancy. Therefore, intranasally administered vaccines may lead to reduced hesitancy and increased vaccine compliance relative to injected vaccines. Additionally, the nasal formulations described herein may be stable for extended periods of time at temperatures above freezing, simplifying transport, storage, and distribution of vaccines relative to formulations that must be stored at frozen or at temperatures well below freezing (e.g., at ultra-cold temperatures from about −90° C. to about −60° C.).

The compositions described herein may confer viral immunity upon administration to a subject. Immunity may provide a number of advantages to the subject resulting from an enhanced immune response. In addition to reducing the chance of becoming infected with the virus once exposed, enhanced immunity may also decrease the severity of symptoms and increasing the likelihood that an infection is asymptomatic in the event that the subject does become infected with the virus. Furthermore, enhanced immunity may decrease the viral load in the subject and decrease the risk of spreading the virus to others upon exposure.

The viral vaccine compositions described herein may be monovalent (e.g., containing a single viral antigen species) or multivalent (e.g., containing two or more viral antigen species). A multivalent vaccine may provide an additional advantage of conferring immunity to multiple viral strains, variants, or lineages. Additionally, a multivalent vaccine containing multiple antigens for a single virus may retain efficacy against mutated strains by targeting multiple features of the virus. For example, a multivalent vaccine containing two or more antigens from a SARS-CoV-2 alpha variant may confer stronger immunity to a SARS-CoV-2 delta variant than a monovalent vaccine containing a single antigen from the SARS-CoV-2 alpha variant. A monovalent or multivalent vaccine may contain a conformation-specific antigen (e.g. a protein antigen in a prefusion conformation or a postfusion conformation). In some embodiments, an additional protein, small molecule, or antigen is included in the vaccine composition to stabilize a conformation-specific antigen in the preferred conformation. Inoculating a subject with a conformation-specific antigen may improve vaccine efficacy by training the subject's immune system to recognize one or more functional conformations of a virus. For example, inoculating a subject with a vaccine containing a coronavirus prefusion S protein complex and a coronavirus postfusion S protein complex may train a subject's immune system to recognize a coronavirus before and after fusing with a host cell, conferring improved immunity over a vaccine lacking a conformation-specific antigen.

Additionally, vaccine compositions described herein further comprising a stimulator of interferon genes (STING) agonist may confer stronger immunity than a composition lacking a STING agonist by increasing expression of pro-inflammatory cytokines, activating TBK-IRF-3, or inducing expression of INF-β, thereby further stimulating the immune response of the vaccine recipient. A STING agonist included in a vaccine composition may include a small molecule STING agonist, such as a cyclic dinucleotide or a synthetic small molecule STING agonist. The STING agonist may be encapsulated in a lipid nanoparticle or liposome to facilitate delivery.

A viral vaccine of the present disclosure may contain a virion, a component of a virion (e.g., a protein, a lipid, a carbohydrate, or a nucleic acid), or a nucleic acid sequence encoding a component of the virion. The virion or the component of the virion may function as an antigen to trigger production of antibodies against the virion or the component of the virion upon administration of the viral vaccine to a subject. The subject may gain sustained immunity to the virus due to more rapid recognition and immune response to the virus upon subsequent exposure to the virus. The more rapid recognition and immune response may lead to reduced risk in the subject of contracting the virus upon expose following inoculation. Administration of a composition of the present disclosure to a subject (e.g., a human subject or a mammalian subject) may increase immunity of the subject against the one or more viral infections by triggering an immune response in the subject. Administration of the composition to the subject may result in sustained immunity (e.g., mucosal immunity, humoral immunity, or systemic immunity) against the one or more viruses. In some embodiments, a viral vaccine of the present disclosure may be formulated for nasal delivery and delivered to a subject intranasally. A viral vaccine (e.g., a vaccine formulated for nasal delivery) may be a monovalent vaccine or a multivalent vaccine. In some embodiments, the viral vaccine may further comprise a STING agonist to increase an immune response in a vaccine recipient.

### Viral Antigens

Viral vaccine compositions of the present disclosure may contain one or more viral antigens associated with one or more viral infections. A viral antigen may trigger an immune response in a subject upon administration of the viral vaccine to the subject, resulting in a sustained immune response to and rapid identification of the viral antigen upon exposure to a virus comprising the viral antigen. The viral vaccines of the present disclosure may contain viral antigens associated with one or more respiratory viruses (e.g., SARS-CoV-2, SARS-CoV, MERS-CoV, influenza A, or influenza B). In some embodiments, a viral vaccine of the present disclosure is a monovalent vaccine, containing a single species of viral antigen from a single viral strain or lineage (e.g., SARS-CoV-2, SARS-CoV, MERS-CoV, influenza A H1N1, influenza A H2N2, influenza A H3N2, influenza A H5N1, influenza A H7N7, influenza A H1N2, influenza A H7N9, influenza B Victoria, or influenza B Yamagata). In some embodiments, a monovalent vaccine may contain a single species of viral antigen from a single variant or lineage of a virus (e.g., SARS-CoV-2 lineage B.1.1.7 (alpha variant), SARS-CoV-2 lineage B.1.351 (beta variant), SARS-CoV-2 lineage P.1 (gamma variant), SARS-CoV-2 lineage B.1.617.2 (delta variant)), SARS-CoV-2 lineage B.1.526 (iota variant), or SARS-CoV-2 lineage B.1.617.1 (kappa variant). In some embodiments, a viral vaccine of the present disclosure may be a multivalent vaccine, containing two or more species of viral antigens from one or more viral strains or lineages (e.g., SARS-CoV-2, SARS-CoV, MERS-CoV, influenza A H1N1, influenza A H2N2, influenza A H3N2, influenza A H5N1, influenza A H7N7, influenza A H1N2, influenza A H7N9, influenza B Victoria, or influenza B Yamagata). In some embodiments, a multivalent vaccine may contain two or more species of viral antigens for two or more variants or lineages of a virus (e.g., SARS-CoV-2 lineage B.1.1.7 (alpha variant), SARS-CoV-2 lineage B.1.351 (beta variant), SARS-CoV-2 lineage P.1 (gamma variant), or SARS-CoV-2 lineage B.1.617.2 (delta variant)). For example, a viral vaccine may be a divalent vaccine containing two antigens species from one or more viral strains, variants, or lineages. In another example, a viral vaccine may be a trivalent vaccine containing three antigen species from one or more viral strains or lineages. In another example, a viral vaccine may be a quadrivalent vaccine containing four antigen species from one or more viral strains, variants, or lineages. In another example, a viral vaccine may be a multivalent vaccine containing five, six, seven, eight, nine, ten, or more antigen species from one or more viral strains, variants, or lineages.

In some embodiments, a viral vaccine may contain one or more coronavirus antigens (e.g., one or more antigens from SARS-CoV-2, SARS-CoV, MERS-CoV, or a combination thereof), one or more influenza virus antigens (e.g., one or more antigens from influenza A H1N1, influenza A H2N2, influenza A H3N2, influenza A H5N1, influenza A H7N7, influenza A H1N2, influenza A H7N9, influenza B Victoria, influenza B Yamagata, or a combination thereof), or both. For example, a viral vaccine of the present disclosure may contain one or more of a coronavirus spike (S) protein, a coronavirus envelope (E) protein, a coronavirus membrane (M) protein, a live attenuated influenza A H1N1 virion, a live attenuated influenza A H2N2 virion, a live attenuated influenza A H3N2 virion, a live attenuated influenza A H5N1 virion, a live attenuated influenza A H7N7 virion, a live attenuated influenza A H1N2 virion, a live attenuated influenza A H7N9 virion, an influenza A H1N1 protein, an influenza A H2N2 protein, an influenza A H3N2 protein, an influenza A H5N1 protein, an influenza A H7N7 protein, an influenza A H1N2 protein, an influenza A H7N9 protein, a live attenuated influenza B Victoria virus, a live attenuated influenza B Yamagata virus, an influenza B Victoria protein, an influenza B Yamagata protein, or any combination thereof.

In some embodiments, a viral vaccine composition for inoculation against SARS-CoV-2 may comprise a SARS-CoV-2 prefusion S protein complex and a SARS-CoV-2 postfusion S protein complex. In some embodiments, a viral vaccine composition for inoculation against SARS-CoV-2 and influenza A may comprise one or more SARS-CoV-2 antigens (e.g., one or more of a prefusion complex, a postfusion complex, an S protein, an S1 fragment, an S2 fragment, an E protein, an M protein, or an ORF8 protein) and one or more influenza A antigens (e.g., one or more of a live attenuated influenza A H1N1 virion, a live attenuated influenza A H2N2 virion, a live attenuated influenza A H3N2 virion, a live attenuated influenza A H5N1 virion, a live attenuated influenza A H7N7 virion, a live attenuated influenza A H1N2 virion, a live attenuated influenza A H7N9 virion, an influenza A H1N1 protein, an influenza A H2N2 protein, an influenza A H3N2 protein, an influenza A H5N1 protein, an influenza A H7N7 protein, an influenza A H1N2 protein, or an influenza A H7N9 protein). In some embodiments, a viral vaccine composition for inoculation against SARS-CoV-2 and influenza B may comprise one or more SARS-CoV-2 antigens (e.g., one or more of a prefusion complex, a postfusion complex, an S protein, an S1 fragment, an S2 fragment, an E protein, an M protein, or an ORF8 protein) and one or more influenza B antigens (e.g., one or more of a live attenuated influenza B Victoria virus, a live attenuated influenza B Yamagata virus, an influenza B Victoria protein, or an influenza B Yamagata protein). In some embodiments, a viral vaccine composition for inoculation against SARS-CoV-2, influenza A, and influenza B may comprise one or more SARS-CoV-2 antigens (e.g., one or more of a prefusion complex, a postfusion complex, an S protein, an S1 fragment, an S2 fragment, an E protein, an M protein, or an ORF8 protein), one or more influenza A antigens (e.g., one or more of a live attenuated influenza A H1N1 virion, a live attenuated influenza A H2N2 virion, a live attenuated influenza A H3N2 virion, a live attenuated influenza A H5N1 virion, a live attenuated influenza A H7N7 virion, a live attenuated influenza A H1N2 virion, a live attenuated influenza A H7N9 virion, an influenza A H1N1 protein, an influenza A H2N2 protein, an influenza A H3N2 protein, an influenza A H5N1 protein, an influenza A H7N7 protein, an influenza A H1N2 protein, or an influenza A H7N9 protein), and one or more influenza B antigens (e.g., one or more of a live attenuated influenza B Victoria virus, a live attenuated influenza B Yamagata virus, an influenza B Victoria protein, or an influenza B Yamagata protein).

In some embodiments, an antigen of the present disclosure may be derived from egg. In some embodiments, an antigen of the present disclosure may be derived from cell culture. For example, a virus or a viral antigen may be produced in a cell line including dog kidney cells such as MDCK or cells from a clone of MDCK, MDCK-like cells, MDBK cells, MRC-5 cells, WI-38 cells, COS cells, CHO cells, Hep-2 cells, HeLa cells, LLC-MK2 cells, primary chick kidney (PCK) cell, monkey kidney cells such as AGMK cells including Vero cells, suitable pig cell lines, or any other mammalian cell type suitable for the production of influenza virus or viral antigen for vaccine purposes. A virus or viral antigen may be derived from human cells (e.g., MRC-5 cells or Per-C6 cells), primary cells (e.g., chicken embryo fibroblasts), or avian cell lines.

### Coronavirus Antigens

A viral vaccine composition of the present disclosure may contain one or more antigens from one or more coronaviruses. In some embodiments, a viral vaccine may contain antigens from one or more of SARS-CoV-2, SARS-CoV, or MERS-CoV. In some embodiments, an antigen may be specific to a variant or lineage of a virus (e.g., SARS-CoV-2 lineage B.1.1.7 (alpha variant), SARS-CoV-2 lineage B.1.351 (beta variant), SARS-CoV-2 lineage P.1 (gamma variant), or SARS-CoV-2 lineage B.1.617.2 (delta variant)). In some embodiments, an antigen may bind to antibodies that cross-react with two or more viral variants or lineages (e.g., SARS-CoV-2 lineage B.1.1.7 (alpha variant), SARS-CoV-2 lineage B.1.351 (beta variant), SARS-CoV-2 lineage P.1 (gamma variant), or SARS-CoV-2 lineage B.1.617.2 (delta variant)). An antigen may comprise a live attenuated virion, a protein, a carbohydrate, a lipid, or a nucleic acid. In some embodiments, an antigen may comprise a glycosylated protein. In some embodiments, a viral vaccine of the present disclosure may contain one or more SARS-CoV-2 proteins. For example, a viral vaccine of the present disclosure may contain one or more of a SARS-CoV-2 S protein (e.g., SEQ ID NO: 1, SEQ ID NO: 4, or SEQ ID NO: 8), a fragment of a SARS-CoV-2 S protein (e.g., SEQ ID NO: 2, SEQ ID NO: 3, SEQ ID NO: 5, SEQ ID NO: 9, or SEQ ID NO: 10), a SARS-CoV-2 M protein (e.g., SEQ ID NO: 6), a SARS-CoV-2 E protein (e.g., SEQ ID NO: 7), or a SARS-CoV-2 ORF8 protein, fragment or variant (e.g., SEQ ID NO: 11-SEQ ID NO: 14).

In some embodiments, a SARS-CoV-2 antigen may comprise a protein having at least about 50%, at least about 60%, at least about 70%, at least about 75%, at least about 80%, at least about 85%, at least about 90%, at least about 95%, at least about 97%, at least about 99%, or about 100% sequence identity to any one of SEQ ID NO: 1-SEQ ID NO: 14. For example, a SARS-CoV-2 S protein or protein fragment may comprise one or more amino acid substitutions at T19R, T95I, G142D, R158G, L452R, T478K, D614G, P681R, or D950N; one or more amino acid deletions at E156 or F157; or combinations thereof, with respect to SEQ ID NO: 1. A SARS-CoV-2 S protein or protein fragment may further comprise one or more amino acid substitutions at V70F, A222V, W258L, or K417N, or combinations thereof, with respect to SEQ ID NO: 1. For example, an S protein of SEQ ID NO: 8 or an S1 fragment of SEQ ID NO: 9 may further comprise amino acid substitutions at one or more of V70F, A222V, W258L, or K417N. In some embodiments, a SARS-CoV-2 antigen may comprise a fragment of any one of SEQ ID NO: 1-SEQ ID NO: 14, wherein a fragment comprises a length of at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, at least about 1000, at least about 1100, at least about 1200 amino acid residues. In some embodiments, a SARS-CoV-2 antigen may comprise a protein having at least about 50%, at least about 60%, at least about 70%, at least about 75%, at least about 80%, at least about 85%, at least about 90%, at least about 95%, at least about 97%, at least about 99%, or about 100% sequence identity to a fragment of any one of SEQ ID NO: 1-SEQ ID NO: 14, wherein a fragment comprises a length of at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, at least about 1000, at least about 1100, at least about 1200 amino acid residues.

TABLE 1
Exemplary SARS-CoV-2 Proteins

| SEQ ID NO: | Description | Sequence |
|---|---|---|
| SEQ ID NO: 1 | S protein | MFVFLVLLPLVSSQCVNLTTRTQLPPAYTNSFTRGVYY |
| | | PDKVFRSSVLHSTQDLFLPFFSNVTWFHAIHVSGTNGT |
| | | KRFDNPVLPFNDGVYFASTEKSNIIRGWIFGTTLDSKTQ |
| | | SLLIVNNATNVVIKVCEFQFCNDPFLGVYYHKNNKSW |
| | | MESEFRVYSSANNCTFEYVSQPFLMDLEGKQGNFKNL |
| | | REFVFKNIDGYFKIYSKHTPINLVRDLPQGFSALEPLVD |
| | | LPIGINITRFQTLLALHRSYLTPGDSSSGWTAGAAAYYV |
| | | GYLQPRTFLLKYNENGTITDAVDCALDPLSETKCTLKS |
| | | FTVEKGIYQTSNFRVQPTESIVRFPNITNLCPFGEVFNAT |
| | | RFASVYAWNRKRISNCVADYSVLYNSASFSTFKCYGV |
| | | SPTKLNDLCFTNVYADSFVIRGDEVRQIAPGQTGKIAD |
| | | YNYKLPDDFTGCVIAWNSNNLDSKVGGNYNYLYRLF |
| | | RKSNLKPFERDISTEIYQAGSTPCNGVEGFNCYFPLQSY |
| | | GFQPTNGVGYQPYRVVVLSFELLHAPATVCGPKKSTN |
| | | LVKNKCVNFNFNGLTGTGVLTESNKKFLPFQQFGRDIA |
| | | DTTDAVRDPQTLEILDITPCSFGGVSVITPGTNTSNQVA |
| | | VLYQDVNCTEVPVAIHADQLTPTWRVYSTGSNVFQTR |
| | | AGCLIGAEHVNNSYECDIPIGAGICASYQTQTNSPRRAR |
| | | SVASQSIIAYTMSLGAENSVAYSNNSIAIPTNFTISVTTEI |
| | | LPVSMTKTSVDCTMYICGDSTECSNLLLQYGSFCTQLN |
| | | RALTGIAVEQDKNTQEVFAQVKQIYKTPPIKDFGGFNF |
| | | SQILPDPSKPSKRSFIEDLLFNKVTLADAGFIKQYGDCL |
| | | GDIAARDLICAQKFNGLTVLPPLLTDEMIAQYTSALLA |
| | | GTITSGWTFGAGAALQIPFAMQMAYRFNGIGVTQNVL |
| | | YENQKLIANQFNSAIGKIQDSLSSTASALGKLQDVVNQ |
| | | NAQALNTLVKQLSSNFGAISSVLNDILSRLDKVEAEVQI |
| | | DRLITGRLQSLQTYVTQQLIRAAEIRASANLAATKMSE |
| | | CVLGQSKRVDFCGKGYHLMSFPQSAPHGVVFLHVTYV |
| | | PAQEKNFTTAPAICHDGKAHFPREGVFVSNGTHWFVT |
| | | QRNFYEPQIITTDNTFVSGNCDVVIGIVNNTVYDPLQPE |
| | | LDSFKEELDKYFKNHTSPDVDLGDISGINASVVNIQKEI |
| | | DRLNEVAKNLNESLIDLQELGKYEQYIKWPWYIWLGFI |
| | | AGLIAIVMVTIMLCCMTSCCSCLKGCCSCGSCCKFDED |
| | | DSEPVLKGVKLHYT |
| SEQ ID NO: 2 | S1 fragment | MFVFLVLLPLVSSQCVNLTTRTQLPPAYTNSFTRGVYY |
| | | PDKVFRSSVLHSTQDLFLPFFSNVTWFHAIHVSGTNGT |
| | | KRFDNPVLPFNDGVYFASTEKSNIIRGWIFGTTLDSKTQ |
| | | SLLIVNNATNVVIKVCEFQFCNDPFLGVYYHKNNKSW |
| | | MESEFRVYSSANNCTFEYVSQPFLMDLEGKQGNFKNL |
| | | REFVFKNIDGYFKIYSKHTPINLVRDLPQGFSALEPLVD |
| | | LPIGINITRFQTLLALHRSYLTPGDSSSGWTAGAAAYYV |
| | | GYLQPRTFLLKYNENGTITDAVDCALDPLSETKCTLKS |
| | | FTVEKGIYQTSNFRVQPTESIVRFPNITNLCPFGEVFNAT |
| | | RFASVYAWNRKRISNCVADYSVLYNSASFSTFKCYGV |
| | | SPTKLNDLCFTNVYADSFVIRGDEVRQIAPGQTGKIAD |
| | | YNYKLPDDFTGCVIAWNSNNLDSKVGGNYNYLYRLF |
| | | RKSNLKPFERDISTEIYQAGSTPCNGVEGFNCYFPLQSY |
| | | GFQPTNGVGYQPYRVVVLSFELLHAPATVCGPKKSTN |
| | | LVKNKCVNFNFNGLTGTGVLTESNKKFLPFQQFGRDIA |
| | | DTTDAVRDPQTLEILDITPCSFGGVSVITPGTNTSNQVA |
| | | VLYQDVNCTEVPVAIHADQLTPTWRVYSTGSNVFQTR |
| | | AGCLIGAEHVNNSYECDIPIGAGICASYQTQTNSPRRAR |
| SEQ ID NO: 3 | S2 fragment | SVASQSIIAYTMSLGAENSVAYSNNSIAIPTNFTISVTTEI |
| | | LPVSMTKTSVDCTMYICGDSTECSNLLLQYGSFCTQLN |
| | | RALTGIAVEQDKNTQEVFAQVKQIYKTPPIKDFGGFNF |
| | | SQILPDPSKPSKRSFIEDLLFNKVTLADAGFIKQYGDCL |
| | | GDIAARDLICAQKFNGLTVLPPLLTDEMIAQYTSALLA |
| | | GTITSGWTFGAGAALQIPFAMQMAYRFNGIGVTQNVL |
| | | YENQKLIANQFNSAIGKIQDSLSSTASALGKLQDVVNQ |
| | | NAQALNTLVKQLSSNFGAISSVLNDILSRLDKVEAEVQI |
| | | DRLITGRLQSLQTYVTQQLIRAAEIRASANLAATKMSE |
| | | CVLGQSKRVDFCGKGYHLMSFPQSAPHGVVFLHVTYV |
| | | PAQEKNFTTAPAICHDGKAHFPREGVFVSNGTHWFVT |
| | | QRNFYEPQIITTDNTFVSGNCDVVIGIVNNTVYDPLQPE |
| | | LDSFKEELDKYFKNHTSPDVDLGDISGINASVVNIQKEI |
| | | DRLNEVAKNLNESLIDLQELGKYEQYIKWPWYIWLGFI |
| | | AGLIAIVMVTIMLCCMTSCCSCLKGCCSCGSCCKFDED |
| | | DSEPVLKGVKLHYT |
| SEQ ID NO: 4 | S protein | MFVFLVLLPLVSSQCVNLTTRTQLPPAYTNSFTRGVYY |
| | D614G | PDKVFRSSVLHSTQDLFLPFFSNVTWFHAIHVSGTNGT |
| | | KRFDNPVLPFNDGVYFASTEKSNIIRGWIFGTTLDSKTQ |
| | | SLLIVNNATNVVIKVCEFQFCNDPFLGVYYHKNNKSW |
| | | MESEFRVYSSANNCTFEYVSQPFLMDLEGKQGNFKNL |
| | | REFVFKNIDGYFKIYSKHTPINLVRDLPQGFSALEPLVD |
| | | LPIGINITRFQTLLALHRSYLTPGDSSSGWTAGAAAYYV |
| | | GYLQPRTFLLKYNENGTITDAVDCALDPLSETKCTLKS |
| | | FTVEKGIYQTSNFRVQPTESIVRFPNITNLCPFGEVFNAT |
| | | RFASVYAWNRKRISNCVADYSVLYNSASFSTFKCYGV |
| | | SPTKLNDLCFTNVYADSFVIRGDEVRQIAPGQTGKIAD |
| | | YNYKLPDDFTGCVIAWNSNNLDSKVGGNYNYLYRLF |
| | | RKSNLKPFERDISTEIYQAGSTPCNGVEGFNCYFPLQSY |
| | | GFQPTNGVGYQPYRVVVLSFELLHAPATVCGPKKSTN |
| | | LVKNKCVNFNFNGLTGTGVLTESNKKFLPFQQFGRDIA |
| | | DTTDAVRDPQTLEILDITPCSFGGVSVITPGTNTSNQVA |
| | | VLYQGVNCTEVPVAIHADQLTPTWRVYSTGSNVFQTR |
| | | AGCLIGAEHVNNSYECDIPIGAGICASYQTQTNSPRRAR |
| | | SVASQSIIAYTMSLGAENSVAYSNNSIAIPTNFTISVTTEI |
| | | LPVSMTKTSVDCTMYICGDSTECSNLLLQYGSFCTQLN |
| | | RALTGIAVEQDKNTQEVFAQVKQIYKTPPIKDFGGFNF |
| | | SQILPDPSKPSKRSFIEDLLFNKVTLADAGFIKQYGDCL |
| | | GDIAARDLICAQKFNGLTVLPPLLTDEMIAQYTSALLA |
| | | GTITSGWTFGAGAALQIPFAMQMAYRFNGIGVTQNVL |
| | | YENQKLIANQFNSAIGKIQDSLSSTASALGKLQDVVNQ |
| | | NAQALNTLVKQLSSNFGAISSVLNDILSRLDKVEAEVQI |
| | | DRLITGRLQSLQTYVTQQLIRAAEIRASANLAATKMSE |
| | | CVLGQSKRVDFCGKGYHLMSFPQSAPHGVVFLHVTYV |
| | | PAQEKNFTTAPAICHDGKAHFPREGVFVSNGTHWFVT |
| | | QRNFYEPQIITTDNTFVSGNCDVVIGIVNNTVYDPLQPE |
| | | LDSFKEELDKYFKNHTSPDVDLGDISGINASVVNIQKEI |
| | | DRLNEVAKNLNESLIDLQELGKYEQYIKWPWYIWLGFI |
| | | AGLIAIVMVTIMLCCMTSCCSCLKGCCSCGSCCKFDED |
| | | DSEPVLKGVKLHYT |
| SEQ ID NO: 5 | S1 fragment | MFVFLVLLPLVSSQCVNLTTRTQLPPAYTNSFTRGVYY |
| | D614G | PDKVFRSSVLHSTQDLFLPFFSNVTWFHAIHVSGTNGT |
| | | KRFDNPVLPFNDGVYFASTEKSNIIRGWIFGTTLDSKTQ |
| | | SLLIVNNATNVVIKVCEFQFCNDPFLGVYYHKNNKSW |
| | | MESEFRVYSSANNCTFEYVSQPFLMDLEGKQGNFKNL |
| | | REFVFKNIDGYFKIYSKHTPINLVRDLPQGFSALEPLVD |
| | | LPIGINITRFQTLLALHRSYLTPGDSSSGWTAGAAAYYV |
| | | GYLQPRTFLLKYNENGTITDAVDCALDPLSETKCTLKS |
| | | FTVEKGIYQTSNFRVQPTESIVRFPNITNLCPFGEVFNAT |
| | | RFASVYAWNRKRISNCVADYSVLYNSASFSTFKCYGV |
| | | SPTKLNDLCFTNVYADSFVIRGDEVRQIAPGQTGKIAD |
| | | YNYKLPDDFTGCVIAWNSNNLDSKVGGNYNYLYRLF |
| | | RKSNLKPFERDISTEIYQAGSTPCNGVEGFNCYFPLQSY |
| | | GFQPTNGVGYQPYRVVVLSFELLHAPATVCGPKKSTN |
| | | LVKNKCVNFNFNGLTGTGVLTESNKKFLPFQQFGRDIA |
| | | DTTDAVRDPQTLEILDITPCSFGGVSVITPGTNTSNQVA |
| | | VLYQGVNCTEVPVAIHADQLTPTWRVYSTGSNVFQTR |
| | | AGCLIGAEHVNNSYECDIPIGAGICASYQTQTNSPRRAR |
| SEQ ID NO: 6 | M protein | MADSNGTITVEELKKLLEQWNLVIGFLFLTWICLLQFA |
| | | YANRNRFLYIIKLIFLWLLWPVTLACFVLAAVYRINWI |
| | | TGGIAIAMACLVGLMWLSYFIASFRLFARTRSMWSFNP |
| | | ETNILLNVPLHGTILTRPLLESELVIGAVILRGHLRIAGH |
| | | HLGRCDIKDLPKEITVATSRTLSYYKLGASQRVAGDSG |
| | | FAAYSRYRIGNYKLNTDHSSSSDNIALLVQ |
| SEQ ID NO: 7 | E protein | MYSFVSEETGTLIVNSVLLFLAFVVFLLVTLAILTALRL |
| | | CAYCCNIVNVSLVKPSFYVYSRVKNLNSSRVPDLLV |
| SEQ ID NO: 8 | S protein | MFVFLVLLPLVSSQCVNLRTRTQLPPAYTNSFTRGVYY |
| | delta variant | PDKVFRSSVLHSTQDLFLPFFSNVTWFHAIHVSGTNGT |
| | | KRFDNPVLPFNDGVYFASIEKSNIIRGWIFGTTLDSKTQ |
| | | SLLIVNNATNVVIKVCEFQFCNDPFLDVYYHKNNKSW |
| | | MESGVYSSANNCTFEYVSQPFLMDLEGKQGNFKNLRE |
| | | FVFKNIDGYFKIYSKHTPINLVRDLPQGFSALEPLVDLPI |
| | | GINITRFQTLLALHRSYLTPGDSSSGWTAGAAAYYVGY |
| | | LQPRTFLLKYNENGTITDAVDCALDPLSETKCTLKSFT |
| | | VEKGIYQTSNFRVQPTESIVRFPNITNLCPFGEVFNATRF |
| | | ASVYAWNRKRISNCVADYSVLYNSASFSTFKCYGVSP |
| | | TKLNDLCFTNVYADSFVIRGDEVRQIAPGQTGKIADYN |
| | | YKLPDDFTGCVIAWNSNNLDSKVGGNYNYRYRLFRKS |
| | | NLKPFERDISTEIYQAGSKPCNGVEGFNCYFPLQSYGFQ |
| | | PTNGVGYQPYRVVVLSFELLHAPATVCGPKKSTNLVK |
| | | NKCVNFNFNGLTGTGVLTESNKKFLPFQQFGRDIADTT |
| | | DAVRDPQTLEILDITPCSFGGVSVITPGTNTSNQVAVLY |
| | | QGVNCTEVPVAIHADQLTPTWRVYSTGSNVFQTRAGC |
| | | LIGAEHVNNSYECDIPIGAGICASYQTQTNSRRRARSVA |
| | | SQSIIAYTMSLGAENSVAYSNNSIAIPTNFTISVTTEILPV |
| | | SMTKTSVDCTMYICGDSTECSNLLLQYGSFCTQLNRAL |
| | | TGIAVEQDKNTQEVFAQVKQIYKTPPIKDFGGFNFSQIL |
| | | PDPSKPSKRSFIEDLLFNKVTLADAGFIKQYGDCLGDIA |
| | | ARDLICAQKFNGLTVLPPLLTDEMIAQYTSALLAGTITS |
| | | GWTFGAGAALQIPFAMQMAYRFNGIGVTQNVLYENQ |
| | | KLIANQFNSAIGKIQDSLSSTASALGKLQNVVNQNAQA |
| | | LNTLVKQLSSNFGAISSVLNDILSRLDKVEAEVQIDRLI |
| | | TGRLQSLQTYVTQQLIRAAEIRASANLAATKMSECVLG |
| | | QSKRVDFCGKGYHLMSFPQSAPHGVVFLHVTYVPAQE |
| | | KNFTTAPAICHDGKAHFPREGVFVSNGTHWFVTQRNF |
| | | YEPQIITTDNTFVSGNCDVVIGIVNNTVYDPLQPELDSF |
| | | KEELDKYFKNHTSPDVDLGDISGINASVVNIQKEIDRLN |
| | | EVAKNLNESLIDLQELGKYEQYIKWPWYIWLGFIAGLI |
| | | AIVMVTIMLCCMTSCCSCLKGCCSCGSCCKFDEDDSEP |
| | | VLKGVKLHYT |
| SEQ ID NO: 9 | S1 fragment | MFVFLVLLPLVSSQCVNLRTRTQLPPAYTNSFTRGVYY |
| | delta variant | PDKVFRSSVLHSTQDLFLPFFSNVTWFHAIHVSGTNGT |
| | | KRFDNPVLPFNDGVYFASIEKSNIIRGWIFGTTLDSKTQ |
| | | SLLIVNNATNVVIKVCEFQFCNDPFLDVYYHKNNKSW |
| | | MESGVYSSANNCTFEYVSQPFLMDLEGKQGNFKNLRE |
| | | FVFKNIDGYFKIYSKHTPINLVRDLPQGFSALEPLVDLPI |
| | | GINITRFQTLLALHRSYLTPGDSSSGWTAGAAAYYVGY |
| | | LQPRTFLLKYNENGTITDAVDCALDPLSETKCTLKSFT |
| | | VEKGIYQTSNFRVQPTESIVRFPNITNLCPFGEVFNATRF |
| | | ASVYAWNRKRISNCVADYSVLYNSASFSTFKCYGVSP |
| | | TKLNDLCFTNVYADSFVIRGDEVRQIAPGQTGKIADYN |
| | | YKLPDDFTGCVIAWNSNNLDSKVGGNYNYRYRLFRKS |
| | | NLKPFERDISTEIYQAGSKPCNGVEGFNCYFPLQSYGFQ |
| | | PTNGVGYQPYRVVVLSFELLHAPATVCGPKKSTNLVK |
| | | NKCVNFNFNGLTGTGVLTESNKKFLPFQQFGRDIADTT |
| | | DAVRDPQTLEILDITPCSFGGVSVITPGTNTSNQVAVLY |
| | | QGVNCTEVPVAIHADQLTPTWRVYSTGSNVFQTRAGC |
| | | LIGAEHVNNSYECDIPIGAGICASYQTQTNSRRRAR |
| SEQ ID NO: | S2 fragment | SVASQSIIAYTMSLGAENSVAYSNNSIAIPTNFTISVTTEI |
| 10 | delta variant | LPVSMTKTSVDCTMYICGDSTECSNLLLQYGSFCTQLN |
| | | RALTGIAVEQDKNTQEVFAQVKQIYKTPPIKDFGGFNF |
| | | SQILPDPSKPSKRSFIEDLLFNKVTLADAGFIKQYGDCL |
| | | GDIAARDLICAQKFNGLTVLPPLLTDEMIAQYTSALLA |
| | | GTITSGWTFGAGAALQIPFAMQMAYRFNGIGVTQNVL |
| | | YENQKLIANQFNSAIGKIQDSLSSTASALGKLQNVVNQ |
| | | NAQALNTLVKQLSSNFGAISSVLNDILSRLDKVEAEVQI |
| | | DRLITGRLQSLQTYVTQQLIRAAEIRASANLAATKMSE |
| | | CVLGQSKRVDFCGKGYHLMSFPQSAPHGVVFLHVTYV |
| | | PAQEKNFTTAPAICHDGKAHFPREGVFVSNGTHWFVT |
| | | QRNFYEPQIITTDNTFVSGNCDVVIGIVNNTVYDPLQPE |
| | | LDSFKEELDKYFKNHTSPDVDLGDISGINASVVNIQKEI |
| | | DRLNEVAKNLNESLIDLQELGKYEQYIKWPWYIWLGFI |
| | | AGLIAIVMVTIMLCCMTSCCSCLKGCCSCGSCCKFDED |
| | | DSEPVLKGVKLHYT |
| SEQ ID NO: | ORF8 | MKFLVFLGIITTVAAFHQECSLQSCTQHQPYVVDDPCPI |
| 11 | | HFYSKWYIRVGARKSAPLIELCVDEAGSKSPIQYIDIGN |
| | | YTVSCLPFTINCQEPKLGSLVVRCSFYEDFLEYHDVRV |
| | | VLDFI |
| SEQ ID NO: | ORF8 | VDEAGSKS |
| 12 | fragment | |
| SEQ ID NO: | ORF8 | DEAGSKS |
| 13 | fragment | |
| SEQ ID NO: | ORF8 | VLVVL |
| 14 | functional | |
| | motif | |

In some embodiments, a coronavirus protein antigen may be in a particular conformational state and may function as a conformation-specific antigen. For example, a SARS-CoV-2 S protein may be in a prefusion complex or in a postfusion complex. In some embodiments, a SARS-COV-2 S protein prefusion complex may comprise a trimer of full-length S proteins (e.g., a trimer of SEQ ID NO: 1, SEQ ID NO: 4, or a combination thereof). A prefusion complex may comprise three receptor binding domains (e.g., an S1 fragment corresponding to SEQ ID NO: 2 or SEQ ID NO: 5) and three fusion domains (e.g., an S2 fragment corresponding to SEQ ID NO: 3). A prefusion complex may be capable of binding to a human ACE2 receptor. A postfusion complex may comprise a trimer of truncated S proteins (e.g., a trimer of SEQ ID NO: 3, SEQ ID NO: 10, or a combination thereof). In some embodiments, a truncated S protein may be expressed as a truncated protein. In some embodiments, a truncated S protein may be expressed as a full-length S protein and truncated enzymatically (e.g., by furin). A prefusion complex may comprise glycans linked to one or more asparagine residues. In some embodiments, a conformation-specific antigen (e.g., a prefusion complex or a postfusion complex) may be stabilized by an additional protein, small molecule, or antigen included in the vaccine composition to maintain the preferred conformation of the antigen. In some embodiments, the conformation-specific antigen may be covalently modified (e.g., with post-translational modifications or synthetic cross-linking) to stabilize the antigen in the preferred conformation.

In some embodiments, a coronavirus protein antigen may comprise one or more post-translational modifications (e.g., myristoylation, palmitoylation, isoprenylation, acetylation, alkylation, amidation, glycosylation, hydroxylation, succinylation, sulfonation, glycation, biotinylation, or pegylation). In some embodiments, a coronavirus protein antigen may comprise glycosylation of one or more amino acid residues (e.g., arginine, asparagine, cysteine, hydroxylysine, serine, threonine, tyrosine, tryptophan, or combinations thereof). For example, a coronavirus S protein antigen (e.g., a prefusion complex S protein, a postfusion complex S protein, a full-length S protein, or an S2 fragment) may comprise glycosylation at one or more of N1098, N1134, N1158, N1173, or N1194 relative to SEQ ID NO: 1, at one or more of N1098, N1134, N1158, N1173, or N1194 relative to SEQ ID NO: 4, or at one or more of N1096, N1132, N1156, N1171, or N1192, relative to SEQ ID NO: 8.

In some embodiments, a coronavirus antigen may comprise one or more amino acid substitutions, deletions, insertions, or combinations thereof. For example, a coronavirus S protein antigen may comprise one or more amino acid substitutions corresponding to T19R, T95I, G142D, R158G, L452R, T478K, D614G, P681R, or D950N with respect to SEQ ID NO: 1, one or more deletions corresponding to E156− or F157− with respect to SEQ ID NO: 1, or combinations thereof. An example of an S protein containing amino acid substitutions at T19R, T95I, G142D, R158G, L452R, T478K, D614G, P681R, and D950N and deletions a E156− or F157−, with respect to SEQ ID NO: 1, is provided in SEQ ID NO: 1. In some embodiments, an S protein antigen (e.g., an S protein of SEQ ID NO: 8) may further comprise amino acid substitutions corresponding to V70F, A222V, W258L, or K417N with respect to SEQ ID NO: 1, or combinations thereof. An antigen may comprise a fragment of an S protein comprising one or more amino acid substitutions, deletions, insertions, or combinations (e.g., amino acid substitutions at T19R, T95I, G142D, R158G, L452R, T478K, D614G, P681R, D950N, V70F, A222V, W258L, or K417N, or amino acid deletions at E156− or F157−, or combinations thereof with respect to SEQ ID NO: 1). For example, an antigen may comprise an S1 fragment of SEQ ID NO: 9 or an S2 fragment of SEQ ID NO: 10.

In some embodiments, a coronavirus antigen may be an ORF8 antigen. The SARS-CoV-2 open reading frame 8 (ORF8) protein is a 121-amino acid protein comprising an N-terminal signal sequence followed by a predicted Ig-like fold. The SARS-CoV-2 ORF8 protein forms a homodimer connected by a single disulfide bond. Unlike ORF8 found in other coronaviruses, SARS-CoV-2 ORF8 contains solvent solvent-exposed hydrophobic residues that may play a role in SARS-CoV-2 pathogenesis. SARS-CoV-2 ORF8 contains a histone mimic that disrupts chromatin regulation. ORF8 is predicted to be secreted rather than retained in the ER, and ORF8 antibodies are one of the principal markers of a SARS-CoV-2 infection.

The SARS-CoV-2 ORF8 sequence (SEQ ID NO: 11) is highly divergent from other coronavirus ORF8 sequences, sharing less than 20% sequence identity to SARS-CoV ORF8. This divergence in the ORF8 sequence may contribute to the increased virulence of SARS-CoV-2 relative to other coronaviruses since genetic divergence often contributes to increased virulence in new viral strains. The most conserved region in the ORF8 protein of SARS-CoV-2 is “PFTINCQE” (SEQ ID NO: 15) which is present in the catalytic core of the protein. The SARS-CoV-2 ORF8 shares 95% amino acid sequence identity to the RaTG13 ORF8. The SARS-CoV-2 ORF8 shares 95% nucleic acid sequence identity to the RaTG13 ORF8.

ORF8 is a coronavirus accessory protein may interfere with host inflammatory responses and may contribute to immune evasion of SARS-CoV-2. ORF8 modulates adaptive host immunity and innate immune responses by surpassing interferon-mediated antiviral host responses. SARS-CoV ORF8b comprises a functional motif (VLVVL, SEQ ID NO: 14) that may contribute to the induction of host cell stress pathways and activation of macrophages. However, this functional motif is absent from the SARS-CoV-2 ORF8 protein, reducing activation of the host cell stress pathways macrophages, and leading to a reduced host immune response. As a result, the symptoms of SARS-CoV-2 may be linked to ORF8. The ORF8 protein of SARS-CoV-2 mediates immune evasion through down-regulation of major histocompatibility complex (MHC) class I. SARS-CoV-2 ORF8 directly interacts with MHC-I molecules and mediates their down-regulation and impair host cell antigen presenting system. Additionally, ORF8 may selectively target MHC-I molecules for lysosomal degradation via autophagy. Thus, SARS-CoV-2-infected hosts may be less sensitive to lysis by cytotoxic T lymphocytes.

The ORF8 protein of SARS-CoV-2 induced endoplasmic reticulum stress and mediated immune evasion by antagonizing production of interferon B. Both SARS-CoV-2 ORF8 genotypes, ORF8L and ORF8S, induced ER stress pathways, antagonize interferon B production, and decrease the nuclear translocation of IRF3 induced by poly. The SARS-CoV-2 ORF8 mainly acts as an immune-modulator by down-regulating MHC class I molecules, thereby shielding the infected cells against cytotoxic T cells, killing the target cells. The ORF8 also regulates unfolded protein response (UPR) induced due to the ER stress by triggering the ATF-6 activation, thus enhancing the survivability of infected cells. ORF8 may inhibit induction intracellular aggregation, lysosomal stress, and interleukin-mediated inflammatory responses by activating NLRP3 inflammasomes, thereby inhibiting the host immune response to.

SARS-CoV-2 coronaviruses containing an ORF8 deletion are associated with COVID-19 infections with milder symptoms compared to COVID-19 infections caused by SARS-CoV-2 coronaviruses with a wild type ORF8. The 382-nucleotide deletion variant (Δ382) caused the deletion of the entire ORF8 protein. Patients with the Δ382 variant exhibited less severe symptoms, including milder hypoxic conditions and low cytokine activity compared to patients infected with the wild type virus. The Δ382 variant showed reduced viral replication ability in human cells, suggesting that SARS-CoV-2 ORF8 functions in transmission and viral replication efficiency.

A viral vaccine composition of the present disclosure may comprise a coronavirus ORF8 protein (e.g., SEQ ID NO: 11), an immunogenic fragment of a coronavirus ORF8 protein (e.g., a fragment of SEQ ID NO: 11 comprising from 10 to 120 amino acid residues), a nucleic acid (e.g., a DNA molecule or an RNA molecule) encoding a coronavirus ORF8 protein, or a nucleic acid encoding a fragment of a coronavirus ORF8 protein. The coronavirus may be SARS-CoV, SARS-CoV-2, MERS-CoV, HKU1, OC43, or 229E. The coronavirus may be a beta-coronavirus.

In some embodiments, the ORF8 protein comprises a sequence of SEQ ID NO: 11. In some embodiments, the ORF8 protein may be an ORF8 variant comprising a sequence having at least 80%, 85%, 90%, 95%, or 99% sequence identity to SEQ ID NO: 11. In some embodiments, the ORF8 protein comprises a fragment of a sequence having at least 70%, 80%, 85%, 90%, 95%, or 99% sequence identity to SEQ ID NO: 11. In some embodiments, the ORF8 variant may be an ORF8 protein found in a coronavirus variant (e.g., an ORF8 from a SARS-CoV-2 α, β, γ, or δ variant). In some embodiments, the ORF8 variant may be an ORF8 variation found within a coronavirus strain. In some embodiments, an ORF8 variant may be an engineered ORF8. An ORF8 variant may be engineered to have structural homology to wild type ORF8 or to an ORF8 protein found in a coronavirus variant. Structural homology may be determined using bioinformatic software (e.g., ROSETTA, IntFOLD, RaptorX, Biskit, ESyPred3D, Phyre, MODELLER, or the like). Antibodies generated against the engineered ORF8 variant may also recognize and bind wild type ORF8 or an ORF8 from a coronavirus variant.

An ORF8 protein fragment may be an immunogenic fragment of an ORF8 protein capable of triggering an immune response against the ORF8 fragment or full-length ORF8 in a subject. Antibodies generated against the ORF8 fragment may also recognize and bind full-length ORF8. An immunogenic fragment of ORF8 may be a surface-exposed fragment of ORF8. An immunogenic fragment of ORF8 may be identified using bioinformatics software (e.g., ExPASy, Protean, or BLASTN) to identify fragments predicted to have high immunogenicity. In some embodiments, an immunogenic fragment may be identified by selecting for fragments of ORF8 that bind to one or more polyclonal antibodies generated against full-length ORF8. In some embodiments, an ORF8 fragment may be an ORF8 truncation found in a coronavirus strain or variant. In some embodiments, an ORF8 fragment may comprise a sequence of VDEAGSKS (SEQ ID NO: 12) or DEAGSKS (SEQ ID NO: 13).

In some embodiments, a composition may comprise an ORF8 protein, a fragment of an ORF8 protein, or a nucleic acid encoding an ORF8 protein or protein fragment encapsulated by a delivery vehicle. In some embodiments, the delivery vehicle may comprise a lipid capsid, a viral vector, or an attenuated virus. For example, the delivery vehicle may be a lipid nanoparticle, an adenovirus vector, or an attenuated coronavirus. The delivery vehicle may facilitate delivery of the ORF8 protein, protein fragment, or coding sequence to a host cell.

A composition of the present disclosure may be formulated as vaccine for delivery to a subject. For example, a composition may be formulated as an mRNA vaccine comprising an mRNA encoding an ORF8 protein or protein fragment encapsulated in a lipid particle. In another example, a composition may be formulated an adenovirus vaccine comprising a DNA molecule encoding an ORF8 protein or protein fragment encapsulated in an adenovirus vector. In another example, a composition may be formulated as a subunit vaccine comprising an ORF8 protein or protein fragment. The composition may be formulated in combination with a coronavirus spike protein, a fragment of a coronavirus spike protein, a membrane protein or protein fragment, an envelope protein or protein fragment, or a nucleocapsid protein or protein fragment, or a nucleic acid encoding a coronavirus spike protein or spike protein fragment, a membrane protein or protein fragment, an envelope protein or protein fragment, or a nucleocapsid protein or protein fragment. In some embodiments, the composition may be formulated as a supplemental or booster vaccine to boost immunity conferred by a coronavirus vaccine.

### Influenza Virus Antigens

A viral vaccine composition of the present disclosure may contain one or more antigens from one or more influenza viruses. In some embodiments, a viral vaccine may contain antigens from one or more of influenza A H1N1, influenza A H2N2, influenza A H3N2, influenza A H5N1, influenza A H7N7, influenza A H1N2, influenza A H7N9, influenza B Victoria, or influenza B Yamagata. An antigen may comprise a live attenuated virion, a protein, a carbohydrate, a lipid, or a nucleic acid.

In some embodiments, an influenza antigen may comprise a split influenza virus. where virus particles are disrupted with detergents or other reagents to solubilize the lipid envelope. Such split virus vaccines may contain most or all of the virus structural proteins although not necessarily in the same proportions as they occur in the whole virus. In some embodiments, the influenza antigen may comprise an inactivated influenza antigen, such as inactivated whole virus or recombinant virus, a hemagglutinin A (HA) protein or fragment thereof, a neuraminidase (NA) protein or fragment thereof, or an influenza virosome. In some embodiments, the influenza antigen may comprise a live attenuated influenza virus.

In some embodiments, an influenza antigen may be derived from an engineered influenza viral strain. For example, an influenza viral antigen may be derived from ca A/Ann Arbor/6/60 or ca B/Ann Arbor/1/66. In some embodiments, an influenza viral antigen may comprise a protein comprising one or more amino acid substitutions.

### Additional Reagents

A viral vaccine composition of the present disclosure may comprise one or more reagents in addition to the viral antigens. The one or more additional reagents may confer desirable properties on the viral vaccine compositions, including, for example, stability, viscosity, pH, immunogenicity, or moisture content.

### Adjuvants

An adjuvant may be included in a viral vaccine composition to increase the immune response elicited in a subject receiving the viral vaccine. The adjuvant may comprise intrinsic immunomodulatory properties that may potentiate the host antigen-specific immune response within the subject upon administration of the viral vaccine. Inclusion of an adjuvant in the viral vaccine compositions of the present disclosure may increase or prolong the antigen-specific immune response within the subject upon administration compared to viral vaccine compositions lacking an adjuvant. In some embodiments, an adjuvant may increase or prolong the systemic immunity, the humoral immunity, or the mucosal immunity of the subject to the viral antigens upon administration of the viral vaccine relative to administration of a viral vaccine lacking the adjuvant. In some embodiments, an adjuvant may increase or prolong the mucosal immunity of the subject to the viral antigens upon administration of the viral vaccine relative to administration of a viral vaccine lacking the adjuvant.

In some embodiments, the viral vaccine compositions of the present disclosure may comprise an adjuvant that acts as an agonist of the stimulator of interferon genes (STING) pathway. For example, the STING agonist may comprise a cyclic dinucleotide (e.g., cyclic guanosine monophosphate-adenosine monophosphate (2′3′-cGAMP or cGAMP), cyclic adenosine-inosine monophosphate (cIAMP), ADU-S100 synthetic cyclic dinucleotide, 2′^{AL},3′^{TL}-cGAMP cyclic dinucleotide analog). In some embodiments, the STING agonist may comprise a small molecule STING agonist (e.g., 5,6-Dimethylxanthenone-4-acetic acid (DMXAA), flavone-8-acetic acid, 2,7-bis(2-diethylamino ethoxy)fluoren-9-one, 10-carboxymethyl-9-acridanone, dispiro diketopiperzine, benzo[b][1,4]thiazine-6-carboxamide 18 (G10), α-mangostin (19, α-MG), Benzamide 20 (BNBC), dimeric amidobenzimidazoles (diABZIs), bicyclic benzamide, or a benzothiophene derivative). A STING agonist may increase the immune response produced by the viral vaccine composition upon administration by activating increasing expression of pro-inflammatory cytokines, activating TBK-IRF-3, or inducing expression of INF-β.

In some embodiments, an adjuvant may be encapsulated in a lipid nanoparticle or liposome to facilitate delivery of the adjuvant. For example, encapsulation of the adjuvant (e.g., a cyclic dinucleotide) may facilitate delivery of the adjuvant to the cytosol of a host cell. In some embodiments, a lipid nanoparticle or a liposome encapsulating the adjuvant may comprise one or more lipid species. For example, the lipid nanoparticle or the liposome may comprise one or more of 1,2-dipalmitoyl-sn-glycero-3-phosphocholine (DPPC), 1,2-dipalmitoyl-sn-glycero-3-phospho-(1′-rac-glycerol) (DPPG), 1,2-dipalmitoyl-sn-glycero-3-phosphoethanolamine-N-[methoxy(polyethylene glycol)-2000] (DPPE-PEG2000), cholesterol, 1,2-dioleoyl-sn-glycero-3-phosphocholine (DOPC), or 1,2-dioleoyl-sn-glycero-3-phosphoethanolamine (DOPE). In some embodiments, the lipid nanoparticle or the liposome may comprise DPPC, DPPG, Cholesterol, and DPPE-PEG2000 at a molar ratio of 10:1:1:1, respectively.

In some embodiments, the lipid nanoparticle or the liposome encapsulating the adjuvant may be negatively charged. In some embodiments, the lipid nanoparticle or the liposome encapsulating the adjuvant may comprise a zeta potential of no more than about 0 mV, no more than about −5 mV, no more than about −10 mV, no more than about −15 mV, no more than about −20 mV, no more than about −25 mV, no more than about −30 mV, or no more than about −35 mV. In some embodiments, the lipid nanoparticle or the liposome encapsulating the adjuvant may comprise a zeta potential of from about −100 mV to about 0 mV, from about −90 mV to about 0 mV, from about −80 mV to about 0 mV, from about −70 mV to about 0 mV, from about −60 mV to about 0 mV, from about −50 mV to about 0 mV, from about −40 mV to about 0 mV, from about −30 mV to about 0 mV, from about −100 mV to about −10 mV, from about −90 mV to about −10 mV, from about −80 mV to about −10 mV, from about −70 mV to about −10 mV, from about −60 mV to about −10 mV, from about −50 mV to about −10 mV, from about −40 mV to about −10 mV, from about −30 mV to about −10 mV, from about −100 mV to about −20 mV, from about −90 mV to about −20 mV, from about −80 mV to about −20 mV, from about −70 mV to about −20 mV, from about −60 mV to about −20 mV, from about −50 mV to about −20 mV, from about −40 mV to about −20 mV, from about −30 mV to about −20 mV, from about −100 mV to about −30 mV, from about −90 mV to about −30 mV, from about −80 mV to about −30 mV, from about −70 mV to about −30 mV, from about −60 mV to about −30 mV, from about −50 mV to about −30 mV, or from about −40 mV to about −30 mV.

In some embodiments, the lipid nanoparticle or the liposome encapsulating the adjuvant may have a hydrodynamic diameter of from about 20 nm to about 500 nm, from about 30 nm to about 500 nm, from about 40 nm to about 500 nm, from about 50 nm to about 500 nm, from about 60 nm to about 500 nm, from about 70 nm to about 500 nm, from about 80 nm to about 500 nm, from about 90 nm to about 500 nm, from about 20 nm to about 400 nm, from about 30 nm to about 400 nm, from about 40 nm to about 400 nm, from about 50 nm to about 400 nm, from about 60 nm to about 400 nm, from about 70 nm to about 400 nm, from about 80 nm to about 400 nm, from about 90 nm to about 400 nm, from about 20 nm to about 300 nm, from about 30 nm to about 300 nm, from about 40 nm to about 300 nm, from about 50 nm to about 300 nm, from about 60 nm to about 300 nm, from about 70 nm to about 300 nm, from about 80 nm to about 300 nm, from about 90 nm to about 300 nm, from about 20 nm to about 200 nm, from about 30 nm to about 200 nm, from about 40 nm to about 200 nm, from about 50 nm to about 200 nm, from about 60 nm to about 200 nm, from about 70 nm to about 200 nm, from about 80 nm to about 200 nm, from about 90 nm to about 200 nm, from about 20 nm to about 100 nm, from about 30 nm to about 100 nm, from about 40 nm to about 100 nm, from about 50 nm to about 100 nm, from about 60 nm to about 100 nm, from about 70 nm to about 100 nm, from about 80 nm to about 100 nm, or from about 90 nm to about 100 nm.

### Other Reagents

A viral vaccine composition of the present disclosure may comprise a preservative. In some embodiments, a preservative may increase the stability of a composition. For example, a preservative may increase the shelf life of the viral vaccine composition. In some embodiments, a preservative may comprise benzyl alcohol, parabens thimerosal, chlorobutanol, benzethonium chloride, and benzalkonium chloride, or a combination thereof. In some embodiments, the preservative is benzalkonium chloride. A preservative may be present in the composition at a concentration sufficient to increase the stability of the composition without substantially reducing the efficacy of the viral vaccine. In some embodiments, a preservative may in present in a viral vaccine composition of the present disclosure at a concentration of from about 0.002% to about 2.0% by weight of the composition. In some embodiments, a preservative may in present in a viral vaccine composition of the present disclosure at a concentration of from about 0.01% to about 1.0%, from about 0.01% to about 0.1%, or from about 0.01% to about 0.05% by weight of the composition. In some embodiments, a preservative may be present in the viral vaccine composition at a concentration of from about 0.001% to about 1%, from about 0.002% to about 1%, from about 0.003% to about 1%, from about 0.004% to about 1%, from about 0.005% to about 1%, from about 0.01% to about 1%, from about 0.02% to about 1%, from about 0.03% to about 1%, from about 0.04% to about 1%, from about 0.05% to about 1%, from about 0.1% to about 1%, from about 0.2% to about 1%, from about 0.3% to about 1%, from about 0.4% to about 1%, from about 0.5% to about 1%, from about 0.001% to about 0.5%, from about 0.002% to about 0.5%, from about 0.003% to about 0.5%, from about 0.004% to about 0.5%, from about 0.005% to about 0.5%, from about 0.01% to about 0.5%, from about 0.02% to about 0.5%, from about 0.03% to about 0.5%, from about 0.04% to about 0.5%, from about 0.05% to about 0.5%, from about 0.1% to about 0.5%, from about 0.2% to about 0.5%, from about 0.3% to about 0.5%, from about 0.4% to about 0.5%, from about 0.001% to about 0.1%, from about 0.002% to about 0.1%, from about 0.003% to about 0.1%, from about 0.004% to about 0.1%, from about 0.005% to about 0.1%, from about 0.01% to about 0.1%, from about 0.02% to about 0.1%, from about 0.03% to about 0.1%, from about 0.04% to about 0.1%, from about 0.05% to about 0.1%, from about 0.001% to about 0.01%, from about 0.002% to about 0.01%, from about 0.003% to about 0.01%, from about 0.004% to about 0.01%, from about 0.005% to about 0.01%, from about 0.01% to about 0.01%, from about 0.02% to about 0.01%, from about 0.03% to about 0.01%, from about 0.04% to about 0.01%, or from about 0.05% to about 0.01% by weight of the composition.

A viral vaccine composition of the present disclosure may comprise a buffer. A buffer may be added to the composition to maintain a desired pH. For example, a viral vaccine composition of the present disclosure may comprise a pH of from about pH 4 to about pH 8, from about pH 4.5 to about pH 8, from about pH 5 to about pH 8, from about pH 5.5 to about pH 8, from about pH 6 to about pH 8, from about pH 6.5 to about pH 8, from about pH 7 to about pH 8, from about pH 4 to about pH 7, from about pH 4.5 to about pH 7, from about pH 5 to about pH 7, from about pH 5.5 to about pH 7, from about pH 6 to about pH 7, from about pH 6.5 to about pH 7, from about pH 4 to about pH 6.5, from about pH 4.5 to about pH 6.5, from about pH 5 to about pH 6.5, from about pH 5.5 to about pH 6.5, from about pH 6 to about pH 6.5, from about pH 4 to about pH 6, from about pH 4.5 to about pH 6, from about pH 5 to about pH 6, from about pH 5.5 to about pH 6, from about pH 4 to about pH 5.5, from about pH 4.5 to about pH 5.5, from about pH 5 to about pH 5.5, from about pH 4 to about pH 5, or from about pH 4.5 to about pH 5. In some embodiments, the buffer may comprise citric acid, sodium citrate, monopotassium phosphate, disodium phosphate, potassium biphthalate, sodium hydroxide, sodium acetate, acetic acid, or a combination thereof. For example, the buffer may comprise citric acid and sodium citrate.

In some embodiments, a buffer may be present in the viral vaccine composition at a concentration of from about 0.001% to about 1%, from about 0.002% to about 1%, from about 0.003% to about 1%, from about 0.004% to about 1%, from about 0.005% to about 1%, from about 0.01% to about 1%, from about 0.02% to about 1%, from about 0.03% to about 1%, from about 0.04% to about 1%, from about 0.05% to about 1%, from about 0.1% to about 1%, from about 0.2% to about 1%, from about 0.3% to about 1%, from about 0.4% to about 1%, from about 0.5% to about 1%, from about 0.001% to about 0.5%, from about 0.002% to about 0.5%, from about 0.003% to about 0.5%, from about 0.004% to about 0.5%, from about 0.005% to about 0.5%, from about 0.01% to about 0.5%, from about 0.02% to about 0.5%, from about 0.03% to about 0.5%, from about 0.04% to about 0.5%, from about 0.05% to about 0.5%, from about 0.1% to about 0.5%, from about 0.2% to about 0.5%, from about 0.3% to about 0.5%, from about 0.4% to about 0.5%, from about 0.001% to about 0.1%, from about 0.002% to about 0.1%, from about 0.003% to about 0.1%, from about 0.004% to about 0.1%, from about 0.005% to about 0.1%, from about 0.01% to about 0.1%, from about 0.02% to about 0.1%, from about 0.03% to about 0.1%, from about 0.04% to about 0.1%, from about 0.05% to about 0.1%, from about 0.001% to about 0.01%, from about 0.002% to about 0.01%, from about 0.003% to about 0.01%, from about 0.004% to about 0.01%, from about 0.005% to about 0.01%, from about 0.01% to about 0.01%, from about 0.02% to about 0.01%, from about 0.03% to about 0.01%, from about 0.04% to about 0.01%, or from about 0.05% to about 0.01% by weight of the composition.

A viral vaccine composition of the present disclosure may comprise a humectant. A humectant may be added to a composition of the present disclosure retain moisture in the composition or to reduce moisture loss from the composition. The humectant may inhibit drying of mucous membranes of a subject administered the composition. For example, the humectant may prevent drying of nasal or respiratory mucous membranes of a subject upon nasal administration of the composition to the subject. In some embodiments, a humectant may comprise sorbitol, propylene glycol, and glycerin, or a combination thereof. For example, the humectant may comprise propylene glycol. In some embodiments, a humectant may be present in the viral vaccine composition at a concentration of from about 0.01% to about 10%, from about 0.02% to about 10%, from about 0.03% to about 10%, from about 0.04% to about 10%, from about 0.05% to about 10%, from about 0.10% to about 10%, from about 0.2% to about 10%, from about 0.3% to about 10%, from about 0.4% to about 10%, from about 0.5% to about 10%, from about 1% to about 10%, from about 2% to about 10%, from about 3% to about 10%, from about 4% to about 10%, from about 5% to about 10%, from about 0.01% to about 5%, from about 0.02% to about 5%, from about 0.03% to about 5%, from about 0.04% to about 5%, from about 0.05% to about 5%, from about 0.10% to about 5%, from about 0.2% to about 5%, from about 0.3% to about 5%, from about 0.4% to about 5%, from about 0.5% to about 5%, from about 1% to about 5%, from about 2% to about 5%, from about 3% to about 5%, from about 4% to about 5%, from about 0.01% to about 1%, from about 0.02% to about 1%, from about 0.03% to about 1%, from about 0.04% to about 1%, from about 0.05% to about 1%, from about 0.10% to about 1%, from about 0.2% to about 1%, from about 0.3% to about 1%, from about 0.4% to about 1%, from about 0.5% to about 1%, from about 0.01% to about 0.1%, from about 0.02% to about 0.1%, from about 0.03% to about 0.1%, from about 0.04% to about 0.1%, from about 0.05% to about 0.1%, from about 0.1% to about 0.1%, from about 0.2% to about 0.1%, from about 0.3% to about 0.1%, from about 0.4% to about 0.1%, or from about 0.5% to about 0.1% by weight of the composition.

In some embodiments, a viral vaccine composition may comprise additional reagents, such as monosodium glutamate, hydrolyzed porcine gelatin, arginine, sucrose, monobasic potassium phosphate, dibasic potassium phosphate, ovalbumin, gentamicin sulfate, ethylenediaminetetraacetic acid, or combinations thereof. In some embodiments, the composition may be preservative-free.

### Manufacture and Storage of Intranasal Vaccine Compositions

The viral vaccine compositions described herein may be manufactured using a variety of methods. Vaccine compositions containing a live attenuated virus (e.g., a live attenuated coronavirus, a live attenuated influenza virus, or combinations thereof) may be manufactured by replicating the virus in a cell (e.g., an egg). For example, a virus may be replicated in a specific pathogen-free (SPF) egg by inoculating the egg with the virus. The virus may be harvested by collecting the allantoic fluid from the egg, clarifying by filtration, and concentrating the virus by ultracentrifugation. The concentrated virus may then be diluted into a buffered solution (e.g., a solution comprising sucrose and potassium phosphate) and sterile filtered to produce a monovalent bulk strain. Multiple monovalent bulk strains may be replicated and harvested separately and subsequently combined to produce a multivalent bulk vaccine.

Vaccine compositions containing a viral protein or protein fragment antigen may be manufactured by recombinantly expressing the antigen (e.g., a coronavirus spike protein or spike protein fragment) in cell culture. For example, the viral antigen may be synthesized by over-expressing the viral protein or protein fragment a eukaryotic cell culture (e.g., mammalian, yeast, or insect cell culture) or a prokaryotic cell culture (e.g., bacterial cell culture). The protein or protein fragment may be purified, for example via one or more rounds of chromatography, and diluted or dialyzed into a buffered solution (e.g., a solution comprising sucrose and potassium phosphate) to produce a monovalent antigen solution. Multiple monovalent antigen solutions may be combined to produce a multivalent antigen solution.

In some embodiments, liposomes containing a STING agonist (e.g., cGAMP, cIAMP, ADU-S100 synthetic cyclic dinucleotide, 2′^{AL},3′^{TL}-cGAMP cyclic dinucleotide analog, 5,6-Dimethylxanthenone-4-acetic acid (DMXAA), flavone-8-acetic acid, 2,7-bis(2-diethylamino ethoxy)fluoren-9-one, 10-carboxymethyl-9-acridanone, dispiro diketopiperzine, benzo[b][1,4]thiazine-6-carboxamide 18 (G10), α-mangostin (19, α-MG), Benzamide 20 (BNBC), dimeric amidobenzimidazoles (diABZIs), bicyclic benzamide, or a benzothiophene derivative) may be manufactured by mixing lipids (e.g., DPPC, DPPG, cholesterol, DPPE, DPPE-PEG, or combinations thereof) in an organic solution (e.g., chloroform, ethanol, or a combination thereof). The lipids may be dried, for example by evaporation, and the lipid film may be hydrated in a buffered solution containing the STING agonist. The lipids and sting agonist may be mixed at an elevated temperature (e.g., about 65° C.) to solubilize and subjected to freeze-thaw cycles to produce liposomes encapsulating the STING agonist. Unencapsulated STING agonist may be removed, for example by filtration. The STING agonist-containing liposomes may be combined with a monovalent or multivalent antigen solution or live attenuated virus solution to produce a monovalent or multivalent viral vaccine containing a STING agonist.

In some embodiments, a viral vaccine composition (e.g., a viral vaccine formulated for nasal delivery) may be stable at temperatures above freezing. For example, a viral vaccine formulated for nasal delivery may be stable at temperatures of from about 1° C. to about 10° C., or from about 2° C. to about 8° C. In some embodiments, the viral vaccine may be stable at a temperature of up to about 25° C. or up to about 27° C. for up to about 12 hours or up to about 24 hours. In another example, a viral vaccine composition formulated for nasal delivery may be stable at room temperature (e.g., from about 15° C. to about 25° C.). The viral vaccine may be stored at temperatures from just above freezing (e.g., above 0° C.) to about 25° C. for up to about 1 month, 2 months, 3 months, 4 months, 5 months, 6 months, or 1 year.

### Formulations and Methods for Nasal Administration

The viral vaccine compositions provided herein for prevention of a viral infection may be formulated for nasal delivery. Nasal delivery of viral vaccines may be well suited for vaccines targeted respiratory viruses. Administering a vaccine via the nasal passage may result in an enhanced mucosal immune response, for example at mucus membranes in the respiratory tract, relative to other methods of administration (e.g., injection or oral administration). Enhanced mucosal immunity may result in improved immunity at the site of infection (e.g., the lungs) and reduce the risk of infection or the severity of respiratory symptoms in the event of infection. In some embodiments, a composition or viral antigen may be purified, for example by filtration. Filtration may comprise filtration through reverse osmosis membranes, nanofiltration membranes, ultrafiltration membranes, and microfiltration membranes. In some embodiments, a filtration membrane may comprise regenerated cellulose, polyether sulfone, polyvinylidene fluoride (PVDF), and ceramic and metal oxide aggregates, as well as polycarbonate, polypropylene, polyethylene, and PTFE (TEFLON®). In some embodiments, combinations of filtration methods and membrane types may be used. In some embodiments, multiple filter types may be used. A filter may have a pore size about 0.2 microns to about 3.0 microns. In some embodiments, a pre-filter may be used in a filtration process prior to the use of one or more other filters and can remove certain cell debris components from a viral harvest (e.g., host cell debris). A pre-filter may have a pore size that is larger than one or more downstream filters. In some embodiments, a pre-filter has a pore size ranging from about 3 microns to about 20 microns.

In some embodiments, a viral vaccine formulated for nasal delivery may be formulated as an aqueous solution. A formulation for nasal delivery may have a viscosity of no more than about 1200 centipoise (cP), no more than about 1100 cP, no more than about 1000 cP, no more than about 900 cP, or no more than about 800 cP. A formulation for nasal delivery may have a pH of no less than about pH 4 and no more than about pH 6. In some embodiments, a formulation for nasal delivery may have a pH of about 5.

A viral vaccine composition formulated for nasal delivery may be sterile, as measured by a U.S. Pharmacopeia <61> method. A sterile composition may comprise an amount of mesophilic bacteria and fungi capable of growing under aerobic conditions below a maximum acceptable amount. A viral vaccine composition formulated for nasal delivery may comprise a concentration of impurities of less than about 0.10%, less than about 0.09%, less than about 0.08%, less than about 0.07%, less than about 0.06%, or less than about 0.05% by weight relative to an active ingredient in the composition (e.g., relative to a viral protein concentration). A viral vaccine composition formulated for nasal delivery may comprise an amount of impurities of no more than about 1.0 mg per day intake, no more than about 0.9 mg per day intake, no more than about 0.8 mg per day intake, no more than about 0.7 mg per day intake, no more than about 0.6 mg per day intake, or no more than about 0.5 mg per day intake. In some embodiments, impurities may include one or more of synthesis-related impurities, degradation products, heavy metals, or other components not disclosed on a label of the composition.

A composition of the present disclosure may be contained in a sprayer configured for nasal delivery. In some embodiments, the sprayer contains a single dose of the composition. A single dose sprayer may comprise a dose divider configured for administration of a first portion of the dose to a first nostril of a subject and, following removal of the dose divider, administration of a second portion of the dose to a second nostril of the subject. In some embodiments, the sprayer contains multiple doses of the composition. A multi-dose sprayer may be configured to deliver a metered amount of a dose to a nostril of a subject. For example, a multi-dose sprayer may deliver half of a dose per spray, and one spray may be administered to each nostril of the subject. In some embodiments, a composition of the present disclosure may be administered to a subject in a dose of about 0.1, about 0.2 mL, about 0.3 mL, or about 0.4 mL. In some embodiments, have of the dose may be administered per nostril of the subject. In some embodiments, the composition may be delivered to a nostril in a spray plume. The spray plume may contain no less than about 75% or no more than about 125% of a target spray volume. In some embodiments, a target spray volume is half a dose. In some embodiments, a target spray volume is a quarter of a dose.

In some embodiments, administration of a viral vaccine composition of the present disclosure may increase the mucosal immunity of a subject to one or more viruses. Mucosal immunity my lead to improved immunity against a respiratory virus (e.g., SARS-CoV, SARS-CoV-2, and MERS-CoV) as compared to humoral immunity. Nasal administration of the compositions of the present disclosure may increase mucosal immunity relative to administration by other routes (e.g., injection or oral administration).

A viral vaccine composition of the present disclosure may be formulated for nasal delivery and administered via a nasal spray. A nasal spray formulation may be contained within a nasal spray bottle comprising a spray nozzle and a cap. In some embodiments, the bottle may comprise an opaque or semi-opaque material (e.g., opaque plastic or amber glass). In some embodiments, a nasal spray formulation of the present disclosure may be administered to a subject (e.g., a human subject to be inoculated against SARS-CoV-2, influenza, or both) by gently blowing nose to clear nostrils, shaking the bottle, holding the bottle with thumb under the bottle and the spray nozzle between fingers, close off one nostril by pressing fingers against the outside of the nose, insert the spray nozzle into the other nostril until fingers contact the nose, aim the nozzle toward the back of the nose, and spray into the nostril while sniffing gently. In some embodiments, a half dose may be administered to each nostril, or a full dose may be administered to a single nostril.

Aerosol is a product that is packaged under pressure and contains therapeutically active ingredients that are released upon activation of an appropriate valve system. Metered aerosol is a pressurized dosage form comprised of metered dose valves, which allow for the delivery of a uniform quantity of spray upon each activation. Powder aerosol is a product that is packaged under pressure and contains therapeutically active ingredients in the form of a powder, which are released upon activation of an appropriate valve system. Spray aerosol is an aerosol product that utilizes a compressed gas as the propellant to provide the force necessary to expel the product as a wet spray; it generally applicable to solutions of medicinal agents in aqueous solvents. Spray is a liquid minutely divided as by a jet of air or steam. Nasal spray drug products contain therapeutically active ingredients dissolved or suspended in solutions or mixtures of excipients in non-pressurized dispensers. Metered spray is a non-pressurized dosage form consisting of valves that allow the dispensing of a specified quantity of spray upon each activation. Suspension spray is a liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

### Spray Patterns and Plume Geometries for Nasal Delivery

Thorough characterization of the spray's geometry has been found to be a good indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

Plume Height is the measurement from the actuator tip to the point at which the plume angle becomes non-linear because of the breakdown of linear flow. Based on a visual examination of digital images, and to establish a measurement point for width that is consistent with the farthest measurement point of spray pattern. In some embodiments, a height of 30 mm may be used for assessing plume geometry. Major Axis, or Dₘₐₓ, is the largest chord that can be drawn within the fitted spray pattern that crosses the center of mass of the spray pattern (COMw) in base units (mm). Minor Axis is the smallest chord that can be drawn within the fitted spray pattern that crosses the COMw in base units (mm). Ellipticity Ratio, or ovality, is the ratio of the major axis to the minor axis. D₁₀ is the diameter of droplet for which 10% of the total liquid volume of sample consists of droplets of a smaller diameter (μm). D₅₀ is the diameter of droplet for which 50% of the total liquid volume of sample consists of droplets of a smaller diameter (μm), also known as the mass median diameter. D₉₀ is the diameter of droplet for which 90% of the total liquid volume of sample consists of droplets of a smaller diameter (μm). Span is a measurement of the width of the distribution. The smaller the value, the narrower the distribution. Span is calculated as: (D₉₀−D₁₀)D₅₀. Percent relative standard deviation (% RSD) is the standard deviation divided by the mean of the series and multiplied by 100, also known as % CV.

In some embodiments, a nasal spray pump may be used to nasally administer a formulation to inoculate a subject against a viral infection (e.g., a coronavirus infection, an influenza infection, or both). A nasal spray pump may produce a spray pattern with a maximum diameter (Dₘₐₓ) of from about 10 mm to about 50 mm, from about 20 mm to about 50 mm, from about 30 mm to about 50 mm, from about 40 mm to about 50 mm, from about 10 mm to about 40 mm, from about 20 mm to about 40 mm, from about 30 mm to about 40 mm, from about 10 mm to about 30 mm, from about 20 mm to about 30 mm, or from about 10 mm to about 20 mm. A nasal spray pump may produce a spray pattern with an ovality of from about 1 to about 2, from about 1.2 to about 2, from about 1.3 to about 2, from about 1.4 to about 2, from about 1.5 to about 2, from about 1 to about 1.5, from about 1.2 to about 1.5, from about 1.3 to about 1.5, from about 1.4 to about 1.5, from about 1 to about 1.4, from about 1.2 to about 1.4, from about 1.3 to about 1.4, from about 1 to about 1.3, from about 1.2 to about 1.3, from about 1 to about 1.25, from about 1.15 to about 1.25, from about 1.2 to about 1.25, from about 1 to about 1.2, from about 1.15 to about 1.2, or from about 1 to about 1.15. A nasal spray pump may produce a spray pattern with an area of from about 200 mm² to about 800 mm², from about 300 mm² to about 800 mm², from about 400 mm² to about 800 mm², from about 500 mm² to about 800 mm², from about 600 mm² to about 800 mm², from about 700 mm² to about 800 mm², from about 200 mm² to about 700 mm², from about 300 mm² to about 700 mm², from about 400 mm² to about 700 mm², from about 500 mm² to about 700 mm², from about 600 mm² to about 700 mm², from about 200 mm² to about 600 mm², from about 300 mm² to about 600 mm², from about 400 mm² to about 600 mm², from about 500 mm² to about 600 mm², from about 200 mm² to about 500 mm², from about 300 mm² to about 500 mm², from about 400 mm² to about 500 mm², from about 200 mm² to about 400 mm², from about 300 mm² to about 400 mm², or from about 200 mm² to about 300 mm².

In some embodiments, a nasal spray pump may produce a plume geometry with a plume angle of from about 30° to about 70°, from about 40° to about 70°, from about 50° to about 70°, from about 60° to about 70°, from about 30° to about 60°, from about 40° to about 60°, from about 50° to about 60°, from about 30° to about 50°, from about 40° to about 50°, or from about 30° to about 40°. A nasal spray pump may produce a plume geometry with a plume width of from about 15 mm to about 40 mm, from about 20 mm to about 40 mm, from about 25 mm to about 40 mm, from about 30 mm to about 40 mm, from about 35 mm to about 40 mm, from about 15 mm to about 35 mm, from about 20 mm to about 35 mm, from about 25 mm to about 35 mm, from about 30 mm to about 35 mm, from about 15 mm to about 30 mm, from about 20 mm to about 30 mm, from about 25 mm to about 30 mm, from about 15 mm to about 25 mm, from about 20 mm to about 25 mm, or from about 15 mm to about 20 mm.

In some embodiments, a nasal spray pump may produce a droplet size distribution of which the D₁₀ is from about 10 μm to about 30 μm, from about 12 μm to about 30 μm, from about 15 μm to about 30 μm, from about 17 μm to about 30 μm, from about 20 μm to about 30 μm, from about 25 μm to about 30 μm, from about 10 μm to about 25 μm, from about 12 μm to about 25 μm, from about 15 μm to about 25 μm, from about 17 μm to about 25 μm, from about 20 μm to about 25 μm, from about 10 μm to about 20 μm, from about 12 μm to about 20 μm, from about 15 μm to about 20 μm, from about 17 μm to about 20 μm, from about 10 μm to about 17 μm, from about 12 μm to about 17 μm, from about 15 μm to about 17 μm, from about 10 μm to about 15 μm, from about 12 μm to about 15 μm, or from about 10 μm to about 12 μm.

A nasal spray pump may produce a droplet size distribution of which the D₅₀ is from about 25 μm to about 75 μm, from about 30 μm to about 75 μm, from about 35 μm to about 75 μm, from about 40 μm to about 75 μm, from about 45 μm to about 75 μm, from about 50 μm to about 75 μm, from about 60 μm to about 75 μm, from about 70 μm to about 75 μm, from about 25 μm to about 70 μm, from about 30 μm to about 70 μm, from about 35 μm to about 70 μm, from about 40 μm to about 70 μm, from about 45 μm to about 70 μm, from about 50 μm to about 70 μm, from about 60 μm to about 70 μm, from about 25 μm to about 60 μm, from about 30 μm to about 60 μm, from about 35 μm to about 60 μm, from about 40 μm to about 60 μm, from about 45 μm to about 60 μm, from about 50 μm to about 60 μm, from about 25 μm to about 50 μm, from about 30 μm to about 50 μm, from about 35 μm to about 50 μm, from about 40 μm to about 50 μm, from about 45 μm to about 50 μm, from about 25 μm to about 45 μm, from about 30 μm to about 45 μm, from about 35 μm to about 45 μm, from about 40 μm to about 45 μm, from about 25 μm to about 40 μm, from about 30 μm to about 40 μm, from about 35 μm to about 40 μm, from about 25 μm to about 35 μm, from about 30 μm to about 35 μm, or from about 25 μm to about 30 μm.

A nasal spray pump may produce a droplet size distribution of which the D₉₀ is from about 70 μm to about 150 μm, from about 80 μm to about 150 μm, from about 90 μm to about 150 μm, from about 100 μm to about 150 μm, from about 110 μm to about 150 μm, from about 130 μm to about 150 μm, from about 70 μm to about 130 μm, from about 80 μm to about 130 μm, from about 90 μm to about 130 μm, from about 100 μm to about 130 μm, from about 110 μm to about 130 μm, from about 70 μm to about 110 μm, from about 80 μm to about 110 μm, from about 90 μm to about 110 μm, from about 100 μm to about 110 μm, from about 70 μm to about 100 μm, from about 80 μm to about 100 μm, from about 90 μm to about 100 μm, from about 70 μm to about 90 μm, from about 80 μm to about 90 μm, or from about 70 μm to about 80 μm.

A nasal spray pump may produce a droplet size distribution of which the span is from about 1.5 to about 1.9, from about 1.6 to about 1.9, from about 1.65 to about 1.9, from about 1.7 to about 1.9, from about 1.75 to about 1.9, from about 1.8 to about 1.9, from about 1.5 to about 1.8, from about 1.6 to about 1.8, from about 1.65 to about 1.8, from about 1.7 to about 1.8, from about 1.75 to about 1.8, from about 1.5 to about 1.75, from about 1.6 to about 1.75, from about 1.65 to about 1.75, from about 1.7 to about 1.75, from about 1.5 to about 1.7, from about 1.6 to about 1.7, from about 1.65 to about 1.7, from about 1.5 to about 1.65, from about 1.6 to about 1.65, or from about 1.5 to about 1.6.

A nasal spray pump may produce a droplet size distribution of which the percent of volume below 10 μm is from about 0.40% to about 6%, from about 1% to about 6%, from about 2% to about 6%, from about 3% to about 6%, from about 4% to about 6%, from about 5% to about 6%, from about 0.40% to about 5%, from about 1% to about 5%, from about 2% to about 5%, from about 3% to about 5%, from about 4% to about 5%, from about 0.40% to about 4%, from about 1% to about 4%, from about 2% to about 4%, from about 3% to about 4%, from about 0.40% to about 3%, from about 1% to about 3%, from about 2% to about 3%, from about 0.40% to about 2%, from about 1% to about 2%, or from about 0.40% to about 1%. A nasal spray pump may produce a droplet size distribution of which the percent of volume below 10 μm is at least about 0.4%, at least about 1%, at least about 2%, at least about 3%, at least about 4%, or at least about 5%.

### Devices for Nasal Delivery

A number of devices, such as nasal spray pumps, may be used to nasally administer the compositions of the present disclosure to inoculate a subject against a viral infection (e.g., a coronavirus infection, an influenza infection, or both). In some embodiments, a device may be selected for a desired spray pattern, plume geometry, or droplet size. In some embodiments, a device may be selected based on formulation viscosity. For example, a device may be selected to accurately dispense the desired dosage and produce a wide spray pattern when administering a high viscosity formulation. In some embodiments, a device may be selected to reproducibly produce a uniform plume with a desired plume width (e.g., a narrow plume or a wide plume).

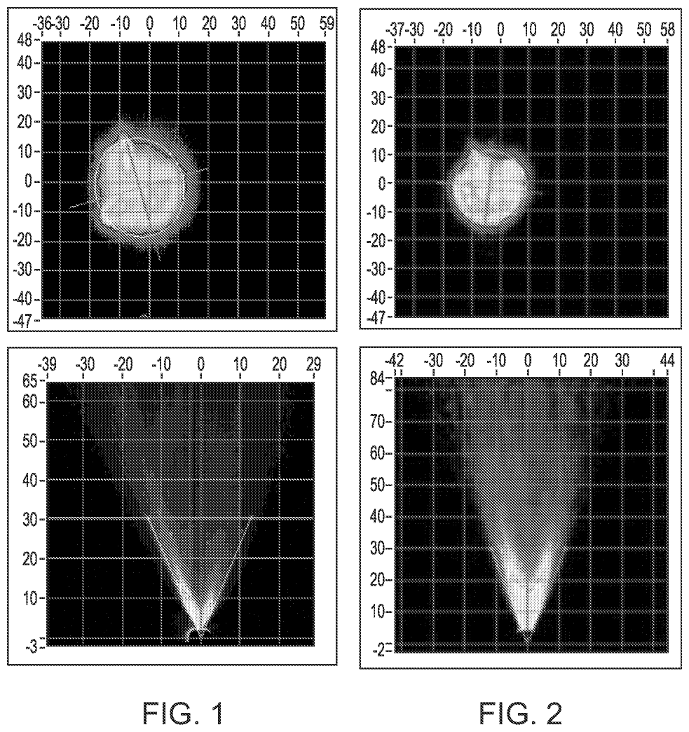

In some embodiments, a device for nasal delivery may delivery a composition of the present disclosure at a spray velocity of from about 90 mm/s to about 110 mm/s. The device may produce a spray pattern with a maximum diameter (Dₘₐₓ) of from about 20 mm to about 32.5 mm, an ovality of from about 1.17 to about 1.38, and an area of about 277 mm² to about 685 mm². The device may produce a plume geometry with a plume angle of from about 37° to about 58.2° and a plume width of from about 20 mm to about 33.5 mm. Examples of spray patterns and plume geometries produced by devices for nasal delivery of formulations for treatment or prevention of a respiratory viral infection are shown in FIG. 1 and FIG. 2. In some embodiments, a device for nasal delivery of a formulation may produce a droplet size distribution of which the D₁₀ is from about 13.1 μm to about 27.2 μm, the D₅₀ is from about 33.9 μm to about 70.9 μm, the D₉₀ is from about 81.1 μm to about 146.5 μm, the span is from about 1.674 μm to about 1.8 μm, and the percent of volume below 10 μm is from about 0.41% to about 4.5%.

FIG. 1 shows the spray pattern and plume geometry of a device that produces a spray pattern with a Dₘₐₓ of about 28.2 mm, an ovality of about 1.219, an area of about 508 mm². The device produces a plume geometry with a plume angle of about 41.9° and a plume width of about 24.5 mm. The device produces droplets with a size distribution of which the D₁₀ is about 19.2 μm, the D₅₀ is about 49.2 μm, the D₉₀ is about 106.6 μm, the span is about 1.8 μm, and the percent of volume below 10 μm is about 1.0%.

FIG. 2 shows the spray pattern and plume geometry of a device that produces a spray pattern with a Dₘₐₓ of about 29 mm, an ovality of about 1.20, an area of about 542 mm². The device produces a plume geometry with a plume angle of about 44° and a plume width of about 24 mm. The device produces droplets with a size distribution of which the D₁₀ is about 18.7 μm, the D₅₀ is about 49.0 μm, the D₉₀ is about 106.5 μm, the span is about 1.792 μm, and the percent of volume below 10 μm is about 1.35%.

### Methods of Preventing a Viral Infection

A viral vaccine composition described here may be administered to a subject to inoculate the subject against a virus, thereby preventing a viral infection. In some embodiments, the viral vaccine may inoculate the subject against a coronavirus (e.g., SARS-CoV, SARS-CoV-2, and MERS-CoV), an influenza virus (e.g., influenza A H1N1, influenza A H2N2, influenza A H3N2, influenza A H5N1, influenza A H7N7, influenza A H1N2, influenza A H7N9, influenza B Victoria, or influenza B Yamagata), or combinations thereof). Preventing a viral infection may comprise reducing the chance that the subject becomes infected with the virus. In some embodiments, preventing a viral infection may comprise reducing the severity of the viral infection in the event that the subject becomes infected. Preventing a viral infection may comprise reducing a viral load within the subject following exposure to the virus. Reducing the severity of the virus may comprise decreasing symptom severity, chance of hospitalization, viral load, or a combination thereof, in the event that the subject becomes infected. In some embodiments, reducing the severity of a viral infection comprises increasing the chance of an infection being asymptomatic.

A viral vaccine may provide a level of immunity to the virus following a single administration of the virus. Immunity to the virus may reduce the likelihood that the subject becomes infected with the virus. Immunity may reduce the viral load within the subject in the event that they become infected with the virus. Immunity may reduce the severity of the viral infection in the event that the subject becomes infected. Immunity may increase the chance of an infection being asymptomatic. In some embodiments, a level of immunity may be increased by administering a second dose of the viral vaccine to the subject. A second dose of the viral vaccine may be administered to the subject about 1 week, about 2 weeks, about 3 weeks, about 4 weeks, about 5 weeks, about 6 weeks, about 7 weeks, or about 8 weeks after a first dose was administered. The second dose is administered from about 3 weeks to about 6 weeks after the first dose was administered. In some embodiments, the viral vaccine may be administered as a booster to a subject that was previously inoculated against the virus. In some embodiments, the subject was previously inoculated with the same vaccine. In some embodiments, the subject was previously inoculated with a different vaccine targeting the same virus. The viral vaccine may be administered as a booster about 3 months, about 4 months, about 5 months, about 6 months, about 7 months, about 8 months, about 9 months, about 10 months, about 11 months, about 12 months, about 14 months, about 16 months, or about 18 months following the previous inoculation. The viral vaccine may be administered about 6 months to about 14 months following the previous inoculation.

The vaccine compositions provided herein may be used to prevent a viral infection in a subject by conferring a level of immunity to the virus. In some embodiments, the level of immunity conferred to the subject by vaccinated with the viral vaccine may be determined by comparing an infection rate among a population vaccinated with the viral vaccine relative to an infection rate among a population that has not been vaccinated. In some embodiments, a viral vaccine composition may confer an immunity of at least about 50%, at least about 60%, at least about 70%, at least about 80%, at least about 85%, at least about 90%, or at least about 95%, relative to an unvaccinated population. In some embodiments, immunity may be maintained at a level of at least 50% for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiments, immunity may be maintained at a level of at least 70% for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiments, immunity may be maintained at a level of at least 80% for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

A second dose of the viral vaccine may confer improved immunity relative to administration of a single dose of the viral vaccine. In some embodiments, a second dose of the viral vaccine may improve immunity by at least about 20%, at least about 40%, at least about 50%, at least about 75%, at least about 90%, or at least about 100% relative to administration of a single dose of the viral vaccine. In some embodiments, a second dose of the viral vaccine may improve immunity by at least about 1.2-fold, at least about 1.5-fold, at least about 1.7-fold, at least about 2-fold, at least about 2.5-fold, or at least about 3-fold relative to administration of a single dose of the viral vaccine. When administered as a booster, the viral vaccine may confer improved immunity relative to administration of the prior vaccine (e.g., prior administration of the same vaccine or prior administration of a different vaccine against the same virus). In some embodiments, a booster of the viral vaccine may improve immunity by at least about 20%, at least about 40%, at least about 50%, at least about 75%, at least about 90%, or at least about 100% relative to administration of the prior vaccine. In some embodiments, a second dose of the viral vaccine may improve immunity by at least about 1.2-fold, at least about 1.5-fold, at least about 1.7-fold, at least about 2-fold, at least about 2.5-fold, or at least about 3-fold relative to administration of the prior vaccine.

In addition to conferring a level of immunity against a virus, administration of the viral vaccine compositions described herein to a subject may reduce the rate of transmission of the virus in the subject in the event of a breakthrough infection. In some embodiments, reduced transmission may result from a reduced viral load in a vaccinated subject relative to an unvaccinated subject upon infection of the subject with the virus. A subject having a breakthrough infection following vaccination with a viral vaccine of the present disclosure may be at least about 20%, at least about 40%, at least about 50%, at least about 75%, at least about 90%, or at least about 100% less likely to transmit the virus relative to an infected unvaccinated individual. A subject having a breakthrough infection following vaccination with a viral vaccine of the present disclosure may be at least about 1.2-fold, at least about 1.5-fold, at least about 1.7-fold, at least about 2-fold, at least about 2.5-fold, or at least about 3-fold less likely to transmit the virus relative to an infected unvaccinated individual.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### EXAMPLES

The invention is further illustrated by the following non-limiting examples.

### Example 1

### Inoculation Against SARS-CoV-2 Using a Bivalent Viral Vaccine

This example describes inoculation of a subject against SARS-CoV-2 using a bivalent viral vaccine. The bivalent viral vaccine contains a SARS-CoV-2 spike protein prefusion complex and a SARS-CoV-2 spike protein postfusion complex. The prefusion complex is a homotrimer of full-length spike proteins containing both the receptor binding S1 fragment and the fusion S2 fragment in a prefusion conformation. The postfusion complex is a homotrimer of truncated spike proteins containing the S2 fragment in a postfusion conformation. The truncated spike proteins in the postfusion complex lack the S1 region. The spike proteins in the postfusion complex are glycosylated with N-linked glycans positioned at one or more of amino acid residues N1098, N1134, N1158, N1173, and N1194, with respect to SEQ ID NO: 1.

The bivalent viral vaccine further contains cyclic guanosine monophosphate-adenosine monophosphate (2′3′-cGAMP) as an adjuvant to enhance the immune response in the subject upon administration of the viral vaccine. 2′3′-cGAMP is a STING pathway agonist. The 2′3′-cGAMP adjuvant is encapsulated in negatively charged lipid nanoparticles containing a molar ratio of 10:1:1:1 of 1,2-Dipalmitoyl-rac-glycero-3-phosphocholine (DPPC), 1,2-dihexadecanoyl-sn-glycero-3-phospho-(1′-rac-glycerol) (DPPG), Cholesterol, and DPPE-PEG2000.

The bivalent viral vaccine is formulated as an aqueous composition for nasal delivery. The nasal delivery formulation contains 2.23% glycerin as a humectant, 0.02% benzalkonium chloride as a preservative, 0.12% sodium citrate as a buffer. The formulation has a pH of 5 and a viscosity of no more than 1000 cPs. The formulation is sterile, as assessed by the U.S. Pharmacopeia <61> method. The formulation contains a concentration of less than 0.10% impurities relative to the concentration of spike protein prefusion and postfusion complexes. Impurities include synthesis-related impurities, degradation products, and heavy metals.

The bivalent viral vaccine is administered to a human subject in the form of a nasal spray, delivering 0.1 mL of the vaccine to each nostril of the subject, for a total dose of 0.2 mL. For subjects less than 9 years of age, a second dose may be administered no less than one week and no more than one month after administration of the first dose. Administration of the viral vaccine triggers an immune response in the subject and reduces the subject's risk of contracting COVID-19.

### Example 2

### Inoculation Against SARS-CoV-2 and Influenza Using a Multivalent Viral Vaccine

This example describes inoculation of a subject against SARS-CoV-2 and influenza using a multivalent viral vaccine. The multivalent viral vaccine contains one or more of a SARS-CoV-2 spike (S) protein, a SARS-CoV-2 spike (S) protein prefusion complex, a SARS-CoV-2 spike (S) protein postfusion complex, a SARS-CoV-2 membrane (M) protein, or a SARS-CoV-2 envelope (E) protein. The prefusion complex is a homotrimer of full-length spike proteins containing both the receptor binding S1 fragment and the fusion S2 fragment in a prefusion conformation. The postfusion complex is a homotrimer of truncated spike proteins containing the S2 fragment in a postfusion conformation. The truncated spike proteins in the postfusion complex lack the S1 region. The spike proteins in the postfusion complex are glycosylated with N-linked glycans positioned at one or more of amino acid residues N1098, N1134, N1158, N1173, and N1194, with respect to SEQ ID NO: 1. Additionally, the multivalent viral vaccine contains an influenza A protein from the H1N1 influenza subtype, the H3N2 subtype, or both and an influenza B protein from the Victoria lineage, the Yamagata lineage, or both.

The multivalent viral vaccine further contains cyclic guanosine monophosphate-adenosine monophosphate (2′3′-cGAMP) as an adjuvant to enhance the immune response in the subject upon administration of the viral vaccine. 2′3′-cGAMP is a STING pathway agonist. The 2′3′-cGAMP adjuvant is encapsulated in negatively charged lipid nanoparticles containing a molar ratio of 10:1:1:1 of 1,2-Dipalmitoyl-rac-glycero-3-phosphocholine (DPPC), 1,2-dihexadecanoyl-sn-glycero-3-phospho-(1′-rac-glycerol) (DPPG), Cholesterol, and DPPE-PEG2000.

The multivalent viral vaccine is formulated as an aqueous composition for nasal delivery. The nasal delivery formulation contains 2.23% glycerin as a humectant, 0.02% benzalkonium chloride as a preservative, 0.12% sodium citrate as a buffer. The formulation has a pH of 5 and a viscosity of no more than 1000 cPs. The formulation is sterile, as assessed by the U.S. Pharmacopeia <61> method. The formulation contains a concentration of less than 0.10% impurities relative to the concentration of spike protein prefusion and postfusion complexes. Impurities include synthesis-related impurities, degradation products, and heavy metals.

The multivalent viral vaccine is administered to a human subject in the form of a nasal spray, delivering 0.1 mL of the vaccine to each nostril of the subject, for a total dose of 0.2 mL. For subjects less than 9 years of age, a second dose may be administered no less than one week and no more than one month after administration of the first dose. Administration of the viral vaccine triggers an immune response in the subject and reduces the subject's risk of contracting COVID-19, Influenza A, and Influenza B. The multivalent viral vaccine is reformulated annually to contain proteins from new strains of SARS-CoV-2, Influenza A, and Influenza B.

### Example 2

### Inoculation Against SARS-CoV-2 Alpha Variant and SARS-CoV-2 Delta Variant Using a Multivalent Viral Vaccine

This example describes inoculation against SARS-CoV-2 alpha variant (SARS-CoV-2 lineage B.1.1.7) and SARS-CoV-2 delta variant (SARS-CoV-2 lineage B.1.617.2) using a multivalent viral vaccine. The multivalent vaccine contains one or more alpha variant spike proteins or spike protein fragments (e.g., one or more of SEQ ID NO: 1, SEQ ID NO: 2, and SEQ ID NO: 3) and one or more delta variant spike proteins or spike protein fragments (e.g., one or more of SEQ ID NO: 8, SEQ ID NO: 9, or SEQ ID NO: 10). The alpha variant spike protein or spike protein fragment is glycosylated with N-linked glycans positioned at one or more of amino acid residues N1098, N1134, N1158, N1173, and N1194, with respect to SEQ ID NO: 1. The delta variant spike protein or spike protein fragment is glycosylated with N-linked glycans positioned at one or more of amino acid residues N1096, N1132, N1156, N1171, and N1192, with respect to SEQ ID NO: 8.

The multivalent viral vaccine further contains cyclic guanosine monophosphate-adenosine monophosphate (2′3′-cGAMP) as an adjuvant to enhance the immune response in the subject upon administration of the viral vaccine. 2′3′-cGAMP is a STING pathway agonist. The 2′3′-cGAMP adjuvant is encapsulated in negatively charged lipid nanoparticles containing a molar ratio of 10:1:1:1 of 1,2-Dipalmitoyl-rac-glycero-3-phosphocholine (DPPC), 1,2-dihexadecanoyl-sn-glycero-3-phospho-(1′-rac-glycerol) (DPPG), Cholesterol, and DPPE-PEG2000.

The multivalent viral vaccine is formulated as an aqueous composition for nasal delivery. The nasal delivery formulation contains 2.23% glycerin as a humectant, 0.02% benzalkonium chloride as a preservative, 0.12% sodium citrate as a buffer. The formulation has a pH of 5 and a viscosity of no more than 1000 cPs. The formulation is sterile, as assessed by the U.S. Pharmacopeia <61> method. The formulation contains a concentration of less than 0.10% impurities relative to the concentration of spike proteins or protein fragments. Impurities include synthesis-related impurities, degradation products, and heavy metals.

The multivalent viral vaccine is administered to a human subject in the form of a nasal spray, delivering 0.1 mL of the vaccine to each nostril of the subject, for a total dose of 0.2 mL. For subjects less than 9 years of age, a second dose may be administered no less than one week and no more than one month after administration of the first dose. Administration of the viral vaccine triggers an immune response in the subject and reduces the subject's risk of contracting COVID-19, Influenza A, and Influenza B. The multivalent viral vaccine is reformulated annually to contain spike proteins or protein fragments from new strains of SARS-CoV-2.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a) a first coronavirus spike protein in a prefusion complex, and
b) a second coronavirus spike in a protein postfusion complex;
wherein the composition is formulated for nasal delivery.

2. The composition of claim 1, further comprising a STING pathway agonist encapsulated in a lipid nanoparticle.

3. The composition of claim 2, wherein the STING pathway agonist comprises a cyclic dinucleotide.

4. The composition of claim 3, wherein the cyclic dinucleotide comprises cyclic guanosine monophosphate-adenosine monophosphate.

5. The composition of any one of claims 2-4, wherein the liposome is negatively charged.

6. The composition of any one of claims 2-5, wherein the liposome comprises an average zeta potential of no greater than 0 mV.

7. The composition of any one of claims 2-6, wherein the liposome has a mean diameter of no less than 30 nm and no greater than 300 nm.

8. The composition of any one of claims 1-7, wherein the postfusion complex comprises one or more N-linked glycans.

9. The composition of any one of claims 1-8, wherein the first coronavirus spike protein comprises one or more N-linked glycans linked to amino acid residues N1098, N1134, N1158, N1173, or N1194, with respect to SEQ ID NO: 1, or combinations thereof.

10. The composition of any one of claims 1-9, wherein the second coronavirus spike protein comprises one or more N-linked glycans linked to amino acid residues N1098, N1134, N1158, N1173, or N1194, with respect to SEQ ID NO: 1, or combinations thereof.

11. The composition of any one of claims 1-10 wherein the first coronavirus spike protein comprises a sequence having at least 90% sequence identity to any one of SEQ ID NO: 1-SEQ ID NO: 5 or SEQ ID NO: 8-SEQ ID NO: 10.

12. The composition of any one of claims 1-11 wherein the second coronavirus spike protein comprises a sequence having at least 90% sequence identity to any one of SEQ ID NO: 1-SEQ ID NO: 5 or SEQ ID NO: 8-SEQ ID NO: 10.

13. A composition comprising:
a) a coronavirus antigen,
b) an influenza A antigen, an influenza B antigen, or both, and
c) a STING pathway agonist encapsulated in a lipid nanoparticle,
wherein the composition is formulated for nasal delivery.

14. The composition of claim 13, wherein the coronavirus antigen is selected from the group consisting of a spike protein, an M protein, an E protein, or an ORF8 protein.

15. The composition of claim 14, wherein the spike protein is in a prefusion complex.

16. The composition of claim 14, wherein the spike protein is in a postfusion complex.

17. The composition of any one of claims 14-16, wherein the spike protein comprises one or more N-linked glycans.

18. The composition of any one of claims 14-17, wherein the spike protein comprises one or more N-linked glycans linked to amino acid residues N1098, N1134, N1158, N1173, or N1194, with respect to SEQ ID NO: 1, or combinations thereof.

19. The composition of any one of claims 14-18, wherein the spike protein comprises a sequence having at least 90% sequence identity to any one of SEQ ID NO: 1-SEQ ID NO: 5 or SEQ ID NO: 8-SEQ ID NO: 10.

20. The composition of any one of claims 14-19, wherein the M protein comprises a sequence having at least 90% sequence identity to SEQ ID NO: 6.

21. The composition of any one of claims 14-20, wherein the E protein comprises a sequence having at least 90% sequence identity to SEQ ID NO: 7.

22. The composition of any one of claims 13-22, wherein the influenza A antigen is from an influenza A subtype selected from H1N1, H3N2, or a combination thereof.

23. The composition of any one of claims 13-22, wherein the influenza B antigen is from an influenza B lineage selected from Victoria, Yamagata, or a combination thereof.

24. The composition of any one of claims 13-23, wherein the STING pathway agonist comprises a cyclic dinucleotide.

25. The composition of claim 24, wherein the cyclic dinucleotide comprises cyclic guanosine monophosphate-adenosine monophosphate.

26. The composition of any one of claims 13-25, wherein the liposome is negatively charged.

27. The composition of any one of claims 13-26, wherein the liposome comprises an average zeta potential of no greater than 0 mV.

28. The composition of any one of claims 13-27, wherein the liposome has a mean diameter of no less than 30 nm and no greater than 300 nm.

29. The composition of any one of claims 1-28, further comprising a preservative.

30. The composition of any one of claims 1-29, further comprising a buffer.

31. The composition of any one of claims 1-30, further comprising a humectant.

32. The composition of any one of claims 1-31, wherein the composition comprises a viscosity of no more than 1000 cPs.

33. A composition comprising:
a) a coronavirus antigen,
b) a STING pathway agonist encapsulated in a lipid nanoparticle,
c) a preservative,
d) a buffer, and
e) a humectant;
wherein the composition comprises a viscosity of no more than 1000 cPs, and wherein the composition is formulated for nasal delivery.

34. The composition of claim 33, wherein the coronavirus antigen is selected from the group consisting of a spike protein, an M protein, or an E protein.

35. The composition of claim 34, wherein the spike protein is in a prefusion complex.

36. The composition of claim 34, wherein the spike protein is in a postfusion complex.

37. The composition of any one of claims 34-36, wherein the spike protein comprises one or more N-linked glycans.

38. The composition of any one of claims 34-37, wherein the spike protein comprises one or more N-linked glycans linked to amino acid residues N1098, N1134, N1158, N1173, or N1194, with respect to SEQ ID NO: 1, or combinations thereof.

39. The composition of any one of claims 34-38, wherein the spike protein comprises a sequence having at least 90% sequence identity to any one of SEQ ID NO: 1-SEQ ID NO: 5 or SEQ ID NO: 8-SEQ ID NO: 10.

40. The composition of any one of claims 34-39, wherein the M protein comprises a sequence having at least 90% sequence identity to SEQ ID NO: 6.

41. The composition of any one of claims 34-40, wherein the E protein comprises a sequence having at least 90% sequence identity to SEQ ID NO: 7.

42. The composition of any one of claims 33-41, wherein the STING pathway agonist comprises a cyclic dinucleotide.

43. The composition of claim 42, wherein the cyclic dinucleotide comprises cyclic guanosine monophosphate-adenosine monophosphate.

44. The composition of any one of claims 33-43, wherein the liposome is negatively charged.

45. The composition of any one of claims 33-44, wherein the liposome comprises an average zeta potential of no greater than 0 mV.

46. The composition of any one of claims 33-45, wherein the liposome has a mean diameter of no less than 30 nm and no greater than 300 nm.

47. The composition of any one of claims 33-46, further comprising an influenza A antigen.

48. The composition of claim 47, wherein the influenza A antigen is from an influenza A subtype selected from H1N1, H3N2, or a combination thereof.

49. The composition of any one of claims 33-48, further comprising an influenza B antigen.

50. The composition of claim 49, wherein the influenza B antigen is from an influenza B lineage selected from Victoria, Yamagata, or a combination thereof.

51. The composition of any one of claims 31-50, wherein the humectant is selected from the group consisting of sorbitol, propylene glycol, and glycerin, and combinations thereof.

52. The composition of any one of claims 31-51, wherein the humectant is propylene glycol.

53. The composition of any one of claims 30-52, wherein the buffer is selected from the group consisting of citric acid, sodium citrate, monopotassium phosphate, disodium phosphate, potassium biphthalate, sodium hydroxide, sodium acetate, acetic acid, and combinations thereof.

54. The composition of any one of claims 30-53, wherein the buffer comprises citric acid and sodium citrate.

55. The composition of any one of claims 29-54, wherein the preservative is selected from the group consisting of benzyl alcohol, parabens thimerosal, chlorobutanol, benzethonium chloride, and benzalkonium chloride, and combinations thereof.

56. The composition of any one of claims 1-55, wherein the composition comprises a pH of no less than 4 and no greater than 6.

57. The composition of any one of claims 1-56, wherein the composition is sterile, as measured by a U.S. Pharmacopeia <61> method.

58. The composition of any one of claims 1-57, comprising an impurity at a concentration of no more than 0.10% relative to the coronavirus protein or no more than 1.0 mg per day intake.

59. The composition of claim 58, wherein the impurity comprises a synthesis-related impurity, a degradation impurity, a heavy metal, or a combination thereof.

60. The composition of any one of claims 1-59, wherein the composition is capable of preventing a viral infection in a subject nasally administered the composition.

61. The composition of any one of claims 1-60, wherein the composition is capable of reducing the severity of a viral infection in a subject nasally administered the composition.

62. The composition of claim 60 or claim 61, wherein the viral infection is a coronavirus infection, an influenza virus infection, or a combination thereof.

63. A method of administering a viral vaccine against a virus to a subject, the method comprising, infusing a nose of the subject with the composition of any one of claims 1-62 in a spray plume, wherein the amount of the composition delivered in a spray plume contains no less than 75% and no more than 125% of a target spray volume.

64. A method of immunizing a subject against a virus, the method comprising nasally administering to the subject the composition of any one of claims 1-62, thereby immunizing the subject.

65. The method of claim 63 or claim 64, wherein administering the composition to the subject increases an immunity to the virus in the subject.

66. The method of any one of claims 63-65, wherein administering the composition to the subject prevents an infection caused by the virus.

67. The method of any one of claims 63-66, wherein administering the composition to the subject reduces a severity of an infection caused by the virus.

68. The method of claim 67, wherein reducing the severity of the infection comprises reducing a risk of hospitalization.

69. The method of claim 67 or claim 68, wherein reducing the severity of the infection comprises increasing a likelihood that the infection is asymptomatic.

70. The method of any one of claims 67-69, wherein reducing the severity of the infection comprises decreasing a severity of a respiratory symptom caused by the virus.

71. The method of any one of claims 63-70, wherein administering the composition to the subject reduces a viral load of the virus in the subject.

72. The method of any one of claims 63-71, wherein administering the composition to the subject increases a mucosal immunity to the virus in the subject.

73. The method of any one of claims 63-72, wherein administering the composition to the subject increases production of antibodies against the virus in the subject.

74. The method of claim 73, wherein the antibodies are produced in a lung of the subject.

75. The method of any one of claims 63-74, wherein the virus is a coronavirus, an influenza virus, or a combination thereof.

76. The method of claim 75, wherein the coronavirus is SARS-CoV-2.

77. The method of claim 76, wherein the SARS-CoV-2 comprises an alpha variant, a beta variant, a gamma variant, a delta variant, iota variant, kappa variant, or a combination thereof.

78. The method of any one of claims 75-77, wherein the influenza virus is an influenza A, an influenza B, or a combination thereof.

79. The method of any one of claims 63-78, comprising:
providing an actuator comprising an actuator tip;
producing an aerosol comprising droplets of the pharmaceutical composition from the actuator tip; and
dispensing the aerosol into a nose of the subject.

80. The method of claim 79, comprising producing a spray pattern ellipticity ratio of no less than 1.0 to not more than 1.4.

81. The method of claim 79 or claim 80, comprising producing a droplet size distribution such that no more than about 5% of the aerosol volume forms droplets that are less than about 10 μm in diameter.

82. The method of any one of claims 79-81, comprising producing a droplet size distribution such that at least 0.4% of the aerosol volume forms droplets that are less than about 10 μm in diameter.

83. The method of any one of claims 79-82, comprising producing a droplet size distribution such that no more than about 50% of the aerosol volume forms droplets that are less than about 26.9 μm in diameter.

84. The method of any one of claims 79-83, comprising producing a droplet size distribution such that a diameter of droplet for which 50% of the aerosol volume forms droplets of smaller diameter is no less than 25 μm and not more than 75 μm.

85. The method of any one of claims 79-84, comprising producing a droplet size distribution such that a diameter of droplet for which 10% of the aerosol volume forms droplets of smaller diameter is no less than 15 μm and not more than 35 μm.

86. The method of any one of claims 79-85, comprising producing a droplet size distribution such that a diameter of droplet for which 90% of the aerosol volume forms droplets of smaller diameter is no less than 70 μm and not more than 150 μm.

87. The method of any one of claims 79-86, comprising producing a spray pattern with a major axis of at least 25 mm and not more than 40 mm and a minor axis of at least 25 mm and not more than 40 mm.
